(19) United States
(12) Patent Application Publication
(10) Pub. No.: US 2014/0302012 A1
(43) Pub. Date: Oct. 9, 2014

(54) Combination Therapies for Treating Hematologic Malignancies Using Pyridopyrimidinone Inhibitors of PI3K/mTOR with Bendamustine and/or Rituximab

(71) Applicants: Arthur DeCillis, Madison, CT (US); Joanne Lager, Hollis, NH (US); Tal Zaks, Newton, MA (US)

(72) Inventors: Arthur DeCillis, Madison, CT (US); Joanne Lager, Hollis, NH (US); Tal Zaks, Newton, MA (US)

(73) Assignees: Sanofi, Paris (FR); Exelixis, Inc., South San Francisco, CA (US)

(21) Appl. No.: 14/126,181

(22) PCT Filed: Jun. 15, 2012
(86) PCT No.: PCT/US12/42582

§ 371 Date: Jun. 10, 2014

Related U.S. Application Data
(60) Provisional application No. 61/497,356, filed on Jun. 15, 2011.
(60) Provisional application No. 61/510,324, filed on Jul. 21, 2011.

(30) Foreign Application Priority Data
Jun. 1, 2012 (FR) 1255114

(51) Int. Cl. A61K 31/519; A61K 39/395; A61K 31/4184

(52) U.S. Cl. CPC A61K 31/519; A61K 31/4184; A61K 39/39558
USPC 424/133.1; 514/264.11

(57) ABSTRACT

The invention provides a method for treating cancers including hematologic malignancies comprising administering a compound of Formula (I): in combination with one or both of bendamustine and rituximab.

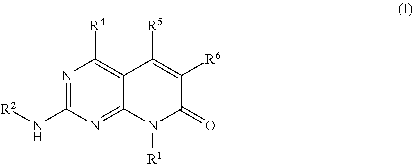

### CROSS-REFERENCE TO RELATED APPLICATIONS

This application claims the benefit of priority of U.S. Provisional Application No. 61/497,356 filed Jun. 15, 2011, U.S. Provisional Application No. 61/510,324 filed Jul. 21, 2011 and French Patent Application No. 1255114 filed Jun. 1, 2012, all of which are incorporated herein by reference.

### BACKGROUND

Non-Hodgkin lymphoma (NHL) is the fifth most common cancer type in the United States with 59,000 new cases per year, and it is associated with high mortality of 41%. Overall 5- and 10-year survival rates for subjects with NHL is 65% and 54%, respectively. B-cell lymphomas are the most common forms, accounting for approximately 85% of all cases and include aggressive and indolent histologic subtypes. Aggressive B-cell lymphomas include Diffuse Large B-call Lymphoma, Mantle Cell Lymphoma and Burkitt's Lymphoma. Follicular Lymphoma, Marginal Zone Lymphomas, Chronic Lymphocytic Leukemia/Small Lymphocytic Lymphoma, and Lymphoplasmacytic Lymphoma are considered indolent B-cell lymphomas. All subtypes of NHL are staged in a similar fashion, and the most often used staging system is the Ann Arbor staging system (American Cancer Society, 2010).

Follicular Lymphoma (FL) is the most common indolent lymphoma comprising approximately 20 to 25% of all newly diagnosed lymphomas (Armitage et al, 1998; International Lymphoma Study Group, 1997). Median survival for all subjects with FL is 7 to 10 years. During the past decade, the ten-year survival rate improved from 52% to 72% (Pulte et al, 2008). FL typically stains positive for CD-10 and also frequently expresses BCL-2 protein as a result of t(14;18). Adverse factors include greater than 4 involved nodal sites, elevated lactate dehydrogenase, older than 60 years of age, advanced stage and hemoglobin less than 12 g/dL (Solal-Celigny et al, 2004). Fcy/RIII polymorphisms may correlate with response to rituximab, and host T cells in the microenvironment may also impact outcome (Relander et al, 2010).

Marginal Zone Lymphomas (MZLs) are a rare, heterogeneous group of disorders consisting of extranodal MZL (MALT lymphoma), nodal MZL and splenic MZL (NCCN, 2010). MALT lymphomas account for 5 to 7% of all NHLs and can occur in gastric or nongastric sites, with the gastric wall being the most common site (ILSG, 1997; Morton et al, 2006). Lymphoplasmacytic lymphoma, also termed Waldenstrom's magroglobulinemia is characterized by excess lyphmoplasmacytic cells in the bone marrow, hyperproduction of immunoglobulin M (IgM) and involvement of visceral organs, including liver and spleen (NCCN, 2010).

Chronic Lymphocytic Leukemia/Small Lymphocytic Lymphoma (CLL/SLL accounts for 35% of adult leukemias and has a variable natural history with survival times of 2 to 20 years (ACS, 2010). It is a B-cell monoclonal disorder characterized by a progressive accumulation of functionally incompetent lymphocytes in the bone marrow, peripheral blood and lymph nodes. CLL/SLL cell immunophenotypes include CD19, CDA, CD20, CD23 expression and low levels of surface immunoglobulin. Karyotypic abnormalities and somatic hypennutation (SHM) in the immunoglobulin heavy chain variable region (IgVH) gene are detectable by fluorescent in situ hybridization (FISH) in 80% of subjects and are the most predictive markers of overall disease outcome (NCCN, 2010). The most common cytogenetic abnormality is del(13q), and disease is relatively benign or slowly progressive. The presence of del(11q) is a negative prognostic factor with a median overall survival of 6 to 7 years. Likewise, a del(17p) is an indicator of a very poor prognosis. This subgroup has a median overall survival of only 2 to 3 years and does not appear to have benefited from recent therapeutic advances. Subjects with a non-mutated IgVH gene tend to have steadily progressive disease and require treatment within a few years. Subjects with a mutated IgVH gene experience more indolent diseases requiring less aggressive or no treatment (Hamblin et al, 1999; Sulda, 2010).

Treatment of hematologic malignancies has advanced in recent years. For example, there are now several treatment options for indolent Non-Hodgkin Lymphoma (iNHL) (NCCN, 2010). Some types of lymphoma, however, such as mantle cell lymphoma (MCL) and diffuse large B-cell lymphoma (DLBCL), have aggressive patterns of progression and remain difficult to treat. Mortality, likewise, remains high in leukemia patients with many subjects ineligible for the most effective drugs targeting specific genetic mutations. Furthermore, treatment of relapsed and refractory (R/R) MCL remains challenging.

Thus, there is an ongoing need for clinically effective agents for treating hematologic malignancies.

### SUMMARY

Accordingly, methods are provided for treating cancer comprising administering to a patient in need thereof a therapeutically effective amount of a Compound of Formula I:

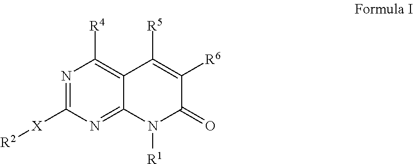

or a pharmaceutically acceptable salt, thereof; or a pharmaceutical composition comprising a therapeutically effective amount of a Compound of Formula I;
in combination with one or both of rituximab and bendamustine, where the Compound of Formula I is that wherein:
R¹ is hydrogen, optionally substituted alkyl, optionally substituted cycloalkyl, optionally substituted cycloalkylalkyl, optionally substituted aryl, optionally substituted arylalkyl, optionally substituted heterocycloalkyl, optionally substituted heterocycloalkylalkyl, optionally substituted heteroaryl or optionally substituted heteroarylalkyl;
R² is hydrogen or alkyl where the alkyl is optionally substituted with 1, 2, 3, 4, or 5 R⁸ groups;
X is —NR³—;
R³ hydrogen;
R⁴ is optionally substituted alkyl;
R⁵ is hydrogen; and
R⁶ is phenyl, acyl, or heteroaryl wherein the phenyl and heteroaryl are optionally substituted with 1, 2, 3, 4, or 5 R⁹ groups;
each R⁸, when present, is independently hydroxy, halo, alkoxy, haloalkoxy, amino, alkylamino, dialkylaminoalkyl, or alkoxyalkylamino; and
each R⁹, when present, is independently halo, alkyl, haloalkyl, alkoxy, haloalkoxy, cyano, amino, alkylamino, dialkylamino, alkoxyalkyl, carboxyalkyl, alkoxycarbonyl, aminoalkyl, cycloalkyl, aryl, arylalkyl, aryloxy, heterocycloalkyl, or heteroaryl and where the cycloalkyl, aryl, heterocycloalkyl, and heteroaryl, each either alone or as part of another group within R⁹, are independently optionally substituted with 1, 2, 3, or 4 groups selected from halo, alkyl, haloalkyl, hydroxy, alkoxy, haloalkxy, amino, alkylamino, and dialkylamino.

In one embodiment, the cancer is a hematologic malignancy which is selected from the group consisting of non-Hodgkin lymphoma (NHL), B-cell lymphomas, including Diffuse Large B-call Lymphoma (DLBCL), Mantle Cell Lymphoma (MCL), Burkitt's Lymphoma, Follicular Lymphoma (FL), Marginal Zone Lymphomas (MZL), Chronic Lymphocytic Leukemia (CLL)/Small Lymphocytic Lymphoma, and Lymphoplasmacytic Lymphoma.

In another embodiment, the hematologic malignancy is relapsed or refractory indolent B-cell Non-Hodgkin Lymphoma and Chronic Lymphocytic Leukemia.

In another embodiment, the compound of Formula I is administered in combination with bendamustine.

In another embodiment, the compound of Formula I is administered in combination with rituximab.

In another embodiment, the compound of Formula I is administered in combination with bendamustine and rituximab.

In one aspect, methods are provided for treating a hematologic malignancy in a patient, the methods comprising administering to the patient an effective amount of (a) 2-amino-8-ethyl-4-methyl-6-(1H-pyrazol-5-yl)pyrido[2,3-d]pyrimidin-7(8H)-one or a pharmaceutically acceptable salt thereof, and either (b) bendamustine or a pharmaceutically acceptable salt thereof or (c) rituximab or (d) a combination of bendamustine or a pharmaceutically acceptable salt thereof and rituximab, wherein the method comprises at least one cycle, wherein the cycle is a period of 28 days, wherein 2-amino-8-ethyl-4-methyl-6-(1H-pyrazol-5-yl)pyrido[2,3-d]pyrimidin-7(8H)-one or the pharmaceutically acceptable salt thereof is administered at about 30 mg BID to about 50 mg BID, and wherein bendamustine or the pharmaceutically acceptable salt thereof is administered at about 70 mg/m² to about 90 mg/m², and rituximab is administered at about 375 mg/m² to about 500 mg/m², wherein the hematologic malignancy is relapsed or refractory indolent B-cell Non-Hodgkin Lymphoma, Mantle Cell Lymphoma or Chronic Lymphocytic Leukemia.

In another aspect, combinations are provided for use in treating a hematologic malignancy, the combination comprising a therapeutically effective amount of (a) 2-amino-8-ethyl-4-methyl-6-(1H-pyrazol-5-yl)pyrido[2,3-d]pyrimidin-7(8H)-one or a pharmaceutically acceptable salt thereof, and either (b) bendamustine or a pharmaceutically acceptable salt thereof or (c) rituximab or (d) a combination of bendamustine or a pharmaceutically acceptable salt thereof and rituximab. In one example, the 2-amino-8-ethyl-4-methyl-6-(1H-pyrazol-5-yl)pyrido[2,3-d]pyrimidin-7(8H)-one or the pharmaceutically acceptable salt thereof is administered at about 30 mg BID to about 50 mg BID, and wherein bendamustine or the pharmaceutically acceptable salt thereof is administered at about 70 mg/m² to about 90 mg/m², and rituximab is administered at about 375 m g/m² to about 500 mg/m², and wherein the hematologic malignancy is relapsed or refractory indolent B-cell Non-Hodgkin Lymphoma, Mantle Cell Lymphoma or Chronic Lymphocytic Leukemia.

### BRIEF DESCRIPTION OF THE DRAWINGS

FIG. 1 provides a plot showing body weight change during, the evaluation of the antitumor activity of Compound A (20 mg/kg) in combination with Bendamustine (3.5 and 7 mg/kg) against human WSU-DLCL2 bearing SCID female mice.

FIG. 2 provides a plot showing antitumor activity Compound A (20 mg/kg) in combination with Bendamustine (3.5 and 7 mg/kg) against human WSU-DLCL2 bearing SCID female mice.

### DETAILED DESCRIPTION

### Abbreviations and Definitions

The following abbreviations and terms have the indicated meanings throughout:

| Abbreviation | Meaning |
|---|---|
| Ac | acetyl |
| br | broad |
| ° C. | degrees Celsius |
| c- | cyclo |
| CBZ | CarboBenZoxy = |
| | benzyloxycarbonyl |
| d | doublet |
| dd | doublet of doublet |
| dt | doublet of triplet |
| DCM | dichloromethane |
| DMA | Dimethylacetamide |
| DME | 1,2-dimethoxyethane |
| DMF | N,N-dimethyl formamide |
| DMSO | dimethyl sulfoxide |
| dppf | 1,1′-bis(diphenylphosphano) |
| | ferrocene |
| EI | Electron Impact ionization |
| g | gram(s) |
| h or hr | hour(s) |
| HPLC | high pressure liquid |
| | chromatography |
| L | liter(s) |
| M | molar or molarity |
| m | Multiplet |
| mg | milligram(s) |
| MHz | megahertz (frequency) |
| Min | minute(s) |
| mL | milliliter(s) |
| μL | microliter(s) |
| μM | Micromole(s) or |
| | micromolar |
| mM | Millimolar |
| mmol | millimole(s) |
| mol | mole(s) |
| MS | mass spectral analysis |
| N | normal or normality |
| nM | Nanomolar |
| NMR | nuclear magnetic |
| | resonance spectroscopy |
| q | Quartet |
| RT | Room temperature |
| s | Singlet |
| t or tr | Triplet |
| TFA | trifluoroacetic acid |
| THF | tetrahydroluran |
| TLC | thin layer chromatography |

The symbol “—” means a single bond, “═” means a double bond, “≡” means a triple bond, “ [symbol] ” means a single or double bond. The symbol “ [symbol] ” refers to a group on a double-bond as occupying either position on the terminus of a double bond to which the symbol is attached; that is, the geometry, E- or Z-, of the double bond is ambiguous. When a group is depicted removed from its parent formula, the “˜” or “ [symbol] ”symbol will be used at the end of the bond which was theoretically cleaved in order to separate the group from its parent structural formula.

When chemical structures are depicted or described, unless explicitly stated otherwise, all carbons are assumed to have hydrogen substitution to conform to a valence of four. For example, in the structure on the left-hand side of the schematic below there are nine hydrogens implied. The nine hydrogens are depicted in the right-hand structure. Sometimes a particular atom in a structure is described in textual formula as having a hydrogen or hydrogens as substitution (expressly defined hydrogen), for example, —CH₂CH₂—. It is understood by one of ordinary skill in the art that the aforementioned descriptive techniques are common in the chemical arts to provide brevity and simplicity to description of otherwise complex structures.

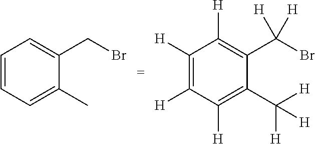

If a group “R” is depicted as “floating” on a ring system, as for example in the formula:

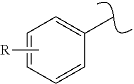

then, unless otherwise defined, a substituent “R” may reside on any atom of the ring system, assuming replacement of a depicted, implied, or expressly defined hydrogen from one of the ring atoms, so long as a stable structure is formed.

If a group “R” is depicted as floating on a fused ring system, as for example in the formulae:

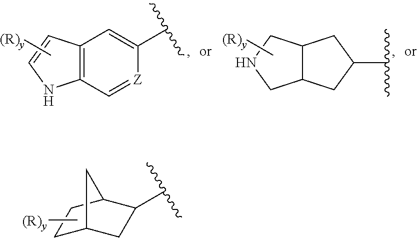

then, unless otherwise, defined, a substituent “R” may reside on any atom of the fused ring system, assuming replacement of a depicted hydrogen (for example the —NH— in the formula above), implied hydrogen (for example as in the formula above, where the hydrogens are not shown but understood to be present), or expressly defined hydrogen (for example where in the formula above, “Z” equals ═CH—) from one of the ring atoms, so long as a stable structure is formed. In the example depicted, the “R” group may reside on either the 5-membered or the 6-membered ring of the fused-ring system. In the formula depicted above, when y is 2 for example, then the two “R's” may reside on any two atoms of the ring system, again assuming each replaces a depicted, implied, or expressly defined hydrogen on the ring.

When a group “R” is depicted as existing on a ring system containing saturated carbons, as for example in the formula:

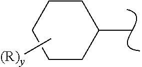

where, in this example, “y” can be more than one, assuming each replaces a currently depicted, implied, or expressly defined hydrogen on the ring; then, unless otherwise defined, where the resulting structure is stable, two “R's” may reside on the same carbon. A simple example is when R is a methyl group; there can exist a geminal dimethyl on a carbon of the depicted ring (an “annular” carbon). In another example, two R's on the same carbon, including that carbon, may form a ring, thus creating a spirocyclic ring (a “spirocyclyl” group) structure with the depicted ring as for example in the formula:

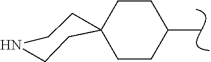

“Acyl” means a —C(O)R radical where R is optionally substituted alkyl, optionally substituted alkenyl, cycloalkyl, cycloalkylalkyl, aryl, aralkyl, heteroaryl, heteroaralkyl, heterocycloalkyl, or heterocycloalkylalkyl, as defined herein, e.g., acetyl, trifluoromethylcarbonyl, or 2-methoxyethylcarbonyl, and the like.

“Acylamino” means a —NRR′ radical where R is hydrogen, hydroxy, alkyl, or alkoxy and R′ is acyl, as defined herein.

“Acyloxy” means an —OR radical where R is acyl, as defined herein, e.g. cyanomethylcarbonyloxy, and the like.

“Administration” and variants thereof (e.g., “administering” a compound) in reference to a compound of the invention means introducing the compound or a prodrug of the compound into the system of the animal in need of treatment. When a compound of the invention or prodrug thereof is provided in combination with one or more other active agents (e.g., bendamustine and/or rituximab), “administration” and its variants are each understood to include concurrent and sequential introduction of the compound or prodrug thereof and other agents.

“Alkenyl” means a means a linear monovalent hydrocarbon radical of one to six carbon atoms or a branched monovalent hydrocarbon radical of three to 6 carbon atoms which radical contains at least one double bond, e.g., ethenyl, propenyl, 1-but-3-enyl, and 1-pent-3-enyl, and the like.

“Alkoxy” means an —OR group where R is alkyl group as defined herein. Examples include methoxy, ethoxy, propoxy, isopropoxy, and the like.

“Alkoxyalkyl” means an alkyl group, as defined herein, substituted with at least one, preferably one, two, or three. alkoxy groups as defined herein. Representative examples include methoxymethyl and the like.

“Alkoxyalkylamino” means an —NRR′ group where R is hydrogen, alkyl, or alkoxyalkyl and R′ is alkoxyalkyl, as defined herein.

“Alkoxyalkylaminoalkyl” means an alkyl group substituted with at least one, specifically one or two, alkoxyalkylamino group(s), as defined herein.

“Alkoxycarbonyl” means a —C(O)R group where R is alkoxy, as defined herein.

“Alkyl” means a linear saturated monovalent hydrocarbon radical of one to six carbon atoms or a branched saturated monovalent hydrocarbon radical of three to 6 carbon atoms, e.g., methyl, ethyl, propyl, 2-propyl, butyl (including all isomeric forms), or pentyl (including all isomeric forms), and the like.

“Alkylamino” means an —NHR group where R is alkyl, as defined herein.

“Alkylaminoalkyl” means an alkyl group substituted with one or two alkylamino groups, as defined herein.

“Alkylaminoalkyloxy” means an —OR group where R is alkylaminoalkyl, as defined herein.

“Alkylcarbonyl” means a —C(O)R group where R is alkyl, as defined herein.

“Alkynyl” means a linear monovalent hydrocarbon radical of one to six carbon atoms or a branched monovalent hydrocarbon radical of three to 6 carbon atoms which radical contains at least one triple bond, e.g., ethynyl, propynyl, butynyl, pentyN-2-yl and the like.

“Amino” means —NH₂.

“Aminoalkyl” means an alkyl group substituted with at least one, specifically one, two or three, amino groups.

“Aminoalkyloxy” means an —OR group where R is aminoalkyl, as defined herein.

“Aryl” means a monovalent six- to fourteen-membered, mono- or bi-carbocyclic ring, wherein the monocyclic ring is aromatic and at least one of the rings in the bicyclic ring is aromatic. Unless stated otherwise, the valency of the group may be located on any atom of any ring within the radical, valency rules permitting. Representative examples include phenyl, naphthyl, and indanyl, and the like.

“Arylalkyl” means an alkyl radical, as defined herein, substituted with one or two aryl groups, as defined herein, e.g., benzyl and phenethyl, and the like.

“Aryloxy” means an —OR group where R is aryl, as defined herein.

“Carboxyalkyl” means an alkyl group, as defined herein, substituted with at least one, specifically one or two, —C(O)OH group(s).

“Cycloalkyl” means a monocyclic or fused bicyclic, saturated or partially unsaturated (but not aromatic), monovalent hydrocarbon radical of three to ten carbon ring atoms. Fused bicyclic hydrocarbon radical includes bridged ring systems. Unless stated otherwise, the valency of the group may be located on any atom of any ring within the radical, valency rules permitting. One or two ring carbon atoms may be replaced by a —C(O)—, —C(S)—, or —C(═NH)— group. More specifically, the term cycloalkyl includes, but is not limited to, cyclopropyl, cyclobutyl, cyclopentyl, cyclohexyl, cyclohexyl, or cyclohex-3-enyl, and the like.

“Cycloalkylalkyl” means an alkyl group substituted with at least one, specifically one or two, cycloalkyl group(s) as defined herein.

“Dialkylamino” means a —NRR′ radical where R and R′ are alkyl as defined herein, or an N-oxide derivative, or a protected derivative thereof, e.g., dimethylamino, diethylamino, N,N-methylpropylamino or N,N-methylethylamino, and the like.

“Dialkylaminoalkyl” means an alkyl group substituted with one or two dialkylamino groups, as defined herein.

“Dialkylaminoalkyloxy” means an —OR group where R is dialkylaminoalkyl, as defined herein. Representative examples include 2-(N,N-diethylamino)-ethyloxy, and the like.

“Fused-polycyclic” or “fused ring system” means a polycyclic ring system that contains bridged or fused rings; that is, where two rings have more than one shared atom in their ring structures. In this application, fused-polycyclics and fused ring systems are not necessarily all aromatic ring systems. Typically, but not necessarily, fused-polycyclics share a vicinal set of atoms, for example naphthalene or 1,2,3,4-tetrahydro-naphthalene. A spiro ring system is not a fused-polycyclic by this definition, but fused polycyclic ring systems of the invention may themselves have Spiro rings attached thereto via a single ring atom of the fused-polycyclic. In some examples, as appreciated by one of ordinary skill in the art, two adjacent groups on an aromatic system may be fused together to form a ring structure. The fused ring structure may contain heteroatoms and may be optionally substituted with one or more groups. It should additionally be noted that saturated carbons of such fused groups (i.e. saturated ring structures) can contain two substitution groups.

“Halogen” or “halo” refers to fluorine, chlorine, bromine or iodine.

“Haloalkoxy” means an —OR′ group where R′ is haloalkyl as defined herein, e.g., trifluoromethoxy or 2,2,2-trifluoroethoxy, and the like.

“Haloalkyl” mean an alkyl group substituted with one or more halogens, specifically one to five halo atoms, e.g., trifluoromethyl, 2-chloroethyl, and 2,2-difluoroethyl, and the like.

“Heteroaryl” means a monocyclic, fused bicyclic, or fused tricyclic, monovalent radical of 5 to 14 ring atoms containing one or more, specifically one, two, three, or four ring heteroatoms independently selected from —O—, —S(O)_{N}— (n is 0, 1, or 2), —N—, —N(R^{x})—, and the remaining ring atoms being carbon, wherein the ring comprising a monocyclic radical is aromatic and wherein at least one of the fused rings comprising a bicyclic or tricyclic radical is aromatic. One or two ring carbon atoms of any nonaromatic rings comprising a bicyclic or tricyclic radical may be replaced by a —C(O)—, —C(S)—, or —C(═NH)— group R^{x} is hydrogen, alkyl, hydroxy, alkoxy, acyl, or alkylsulfonyl. Fused bicyclic radical includes bridged ring systems. Unless stated otherwise, the valency may be located on any atom of any ring of the heteroaryl group, valency rules permitting. When the point of valency is located on the nitrogen, R^{x} is absent. More specifically, the term heteroaryl includes, but is not limited to, 1,2,4-triazolyl, phthalimidyl, pyridinyl, pyrrolyl, imidazolyl, thienyl, furanyl, indolyl, 2,3-dihydro-1H-indolyl (including, for example, 2,3-dihydro-1H-indol-2-yl or 2,3-dihydro-1H-indol-5-yl, and the like), isoindolyl, indolinyl, isoindolinyl, benzimidazolyl, benzodioxol-4-yl, benzofuranyl, cinnolinyl, indolizinyl, naphthyridin-3-yl, phthalazin-3-yl, phthalazin-4-yl, pteridinyl, purinyl, quinazolinyl, quinoxalinyl, tetrazoyl, pyrazolyl, pyrazinyl, pyrimidinyl, pyridazinyl, oxazolyl, isooxazolyl, oxadiazolyl, benzoxazolyl, quinolinyl, isoquinolinyl, tetrahydroisoquinolinyl (including, for example, tetrahydroisoquinolin-4-yl or tetrahydroisoquinolin-6-yl, and the like), pyrrolo[3,2-c]pyridinyl (including, for example, pyrrolo[3,2-c]pyridin-2-yl or pyrrolo[3,2-c]pyridin-7-yl, and the like), benzopyranyl, thiazolyl, isothiazolyl, thiadiazolyl, benzothiazolyl, benzothienyl, and the derivatives thereof, or N-oxide or a protected derivative thereof.

“Heteroarylalkyl” means an alkyl group, as defined herein, substituted with at least one, specifically one or two heteroaryl group(s), as defined herein.

“Heteroatom” refers to O, S, N, or P.

“Heterocycloalkyl” means a saturated or partially unsaturated (but not aromatic) monovalent monocyclic group of 3 to 8 ring atoms or a saturated or partially unsaturated (but not aromatic) monovalent fused bicyclic group of 5 to 12 ring atoms in which one or more, specifically one, two, three, or four ring heteroatoms independently selected from O, S(O)ₙ (n is 0, 1, or 2). N, N(R^{y}) (where R^{y} is hydrogen, alkyl, hydroxy, alkoxy, acyl, or alkylsulfonyl), the remaining ring atoms being carbon. One or two ring carbon atoms may be replaced by a —C(O)—, —C(S)—, or —C(═NH)— group. Fused bicyclic radical includes bridged ring systems. Unless otherwise stated, the valency of the group may be located on any atom of any ring within the radical, valency rules permitting. When the point of valency is located on a nitrogen atom. R^{y} is absent. More specifically the term heterocycloalkyl includes, but is not limited to, azetidinyl, pyrrolidinyl, 2-oxopyrrolidinyl, 2,5-dihydro-1H-pyrrolyl, piperidinyl, 4-piperidonyl, morpholinyl, piperazinyl, 2-oxopiperazinyl, tetrahydropyranyl, 2-oxopiperidinyl, thiomorpholinyl, thiamorpholinyl, perhydroazepinyl, pyrazolidinyl, imidazolinyl, imidazolidinyl, dihydropyridinyl, tetrahydropyridinyl, oxazolinyl, oxazolidinyl, isoxazolidinyl, thiazolinyl, thiazolidinyl, quinuclidinyl, isothiazolidinyl, octahydroindolyl, octahydroisoindolyl, decahydroisoquinolyl, tetrahydrofuryl, and tetrahydropyranyl, and the derivatives thereof and N-oxide or a protected derivative thereof.

“Heterocycloalkylalkyl” means an alkyl radical, as defined herein, substituted with one or two heterocycloalkyl groups, as defined herein, e.g., morpholinylmethyl, N-pyrrolidinylethyl, and 3-(N-azetidinyl)propyl, and the like.

“Heterocycloalkylalkyloxy means an —OR group where R is heterocycloalkylalkyl, as defined herein.

“Saturated bridged ring system” refers to a bicyclic or polycyclic ring system that is not aromatic. Such a system may contain isolated or conjugated unsaturation, but not aromatic or heteroaromatic rings in its core structure (but may have aromatic substitution thereon). For example, hexahydro-furo[3,2-b]furan, 2,3,3a,4,7,7a-hexahydro-1H-indene, 7-aza-bicyclo[2.2.1]heptane, and 1,2,3,4,4a,5,8,8a-octahydro-naphthalene are all included in the class “saturated bridged ring system.

“Spirocyclyl” or “spirocyclic ring” refers to a ring originating from a particular annular carbon of another ring. For example, as depicted below, a ring atom of a saturated bridged ring system (rings B and B′), but not a bridgehead atom, can be a shared atom between the saturated bridged ring system and a spirocyclyl (ring A) attached thereto. A spirocyclyl can be carbocyclic or heteroalicyclic.

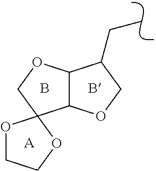

“Optional” or “optionally” means that the subsequently described event or circumstance may or may not occur, and that the description includes instances where said event or circumstance occurs and instances in which it does not. One of ordinary skill in the art would understand that with respect to any molecule described as containing one or more optional substituents, only sterically practical and/or synthetically feasible compounds are meant to be included. “Optionally substituted” refers to all subsequent modifiers in a term. So, for example, in the term “optionally substituted arylC₁₋₈ alkyl,” optional substitution may occur on both the “C₁₋₈ alkyl” portion and the “aryl” portion of the molecule may or may not be substituted. A list of exemplary optional substitutions is presented below in the definition of “substituted.”

“Optionally substituted alkoxy” means an —OR group where R is optionally substituted alkyl, as defined herein.

“Optionally substituted alkyl” means an alkyl radical, as defined herein, optionally substituted with one or more group(s), specifically one, two, three, four, or five groups, independently selected from alkylcarbonyl, alkenylcarbonyl, cycloalkylcarbonyl, alkylcarbonyloxy, alkenylcarbonyloxy, amino, alkylamino, dialkylamino, aminocarbonyl, alkylaminocarbonyl, dialkylaminocarbonyl, cyano, cyanoalkylaminocarbonyl, alkoxy, alkenyloxy, hydroxy, hydroxyalkoxy, halo, carboxy, alkylcarbonylamino, alkylcarbonyloxy, alkyl-S(O)₀₋₂—, alkenyl-S(O)₀₋₂—, aminosulfonyl, alkylaminosulfonyl, dialkylaminosulfonyl, alkylsulfonyl-NR^{c}— (where R^{c} is hydrogen, alkyl, optionally substituted alkenyl, hydroxy, alkoxy, alkenyloxy, or cyanoalkyl), alkylaminocarbonyloxy, dialkylaminocarbonyloxy, alkylaminoalkyloxy, dialkylaminoalkyloxy, alkoxycarbonyl, alkenyloxycarbonyl, alkoxycarbonylamino, alkylaminocarbonylamino, dialkylaminocarbonylamino, alkoxyalkyloxy, and —C(O)NR^{a}R^{b} (where R^{a} and R^{b} are independently hydrogen, alkyl, optionally substituted alkenyl, hydroxy, alkoxy, alkenyloxy, or cyanoalkyl).

“Optionally substituted alkenyl” means an alkyl radical, as defined herein, optionally substituted with one or more group(s), specifically one, two, three, four, or five groups, independently selected from alkylcarbonyl, alkenylcarbonyl, cycloalkylcarbonyl, alkylcarbonyloxy, alkenylcarbonyloxy, amino, alkylamino, dialkylamino, aminocarbonyl, alkylaminocarbonyl, dialkylaminocarbonyl, cyano, cyanoalkylaminocarbonyl, alkoxy, alkenyloxy, hydroxy, hydroxyalkoxy, halo, carboxy, alkylcarbonylamino, alkylcarbonyloxy, alkenyl-S(O)₀₋₂—, aminosulfonyl, alkylaminosulfonyl, dialkylaminosulfonyl, alkylsulfonyl-NR^{c}— (where R^{c} is hydrogen, alkyl, optionally substituted alkenyl, hydroxy, alkoxy, alkenyloxy, or cyanoalkyl), alkylaminocarbonyloxy, dialkylaminocarbonyloxy, alkylaminoalkyloxy, dialkylaminoalkyloxy, alkoxycarbonyl, alkenyloxycarbonyl, alkoxycarbonylamino, alkylaminocarbonylamino, dialkylaminocarbonylamino, alkoxyalkyloxy, and —C(O)NR^{a}R^{b} (where R^{a} and R^{b} are independently hydrogen, alkyl, optionally substituted alkenyl, hydroxy, alkoxy, alkenyloxy, or cyanoalkyl).

“Optionally substituted amino” refers to the group —N(H)R or —N(R)R where each R is independently selected from the group: optionally substituted alkyl, optionally substituted alkoxy, optionally substituted aryl, optionally substituted heterocycloalkyl, optionally substituted heteroaryl, acyl, carboxy, alkoxycarbonyl, —S(O)₂-(optionally substituted alkyl), —S(O)₂-optionally substituted aryl), —S(O)₂-(optionally substituted heterocycloalkyl), —S(O)₂-(optionally substituted heteroaryl), and —S(O)₂-(optionally substituted heteroaryl). For example, “optionally substituted amino” includes diethylamino, methylsulfonylamino, and furanyl-oxy-sulfonamino.

“Optionally substituted aminoalkyl” means an alkyl group, as defined herein, substituted with at least one, specifically one or two, optionally substituted amino group(s), as defined herein.

“Optionally substituted aryl” means an aryl group, as defined herein, optionally substituted with one, two, or three substituents independently selected from acyl, acylamino, acyloxy, optionally substituted alkyl, optionally substituted alkenyl, alkoxy, alkenyloxy, halo, hydroxy, alkoxycarbonyl, alkenyloxycarbonyl, amino, alkylamino, dialkylamino, nitro, aminocarbonyl, alkylaminocarbonyl, dialkylaminocarbonyl, carboxy, cyano, alkylthio, alkylsulfinyl, alkylsulfonyl, aminosulfonyl, alkylaminosulfonyl, dialkylaminosulfonyl, alkylsulfonylamino, aminoalkoxy, or aryl is pentafluorophenyl. Within the optional substituents on “aryl”, the alkyl and alkenyl, either alone or as part of another group (including, for example, the alkyl in alkoxycarbonyl), are independently optionally substituted with One, two, three, four, or five halo.

“Optionally substituted arylalkyl” means an alkyl group, as defined herein, substituted with optionally substituted aryl, as defined herein.

“Optionally substituted cycloalkyl” means a cycloalkyl group, as defined herein, substituted with one, two, or three groups independently selected from acyl, acyloxy, acylamino, optionally substituted alkyl, optionally substituted alkenyl, alkoxy, alkenyloxy, alkoxycarbonyl, alkenyloxycarbonyl, alkylthio, alkylsulfinyl, alkylsulfonyl, aminosulfonyl, alkylaminosulfonyl, dialkylaminosulfonyl, alkylsulfonylamino, halo, hydroxy, amino, alkylamino, dialkylamino, aminocarbonyl, alkylaminocarbonyl, dialkylaminocarbonyl, nitro, alkoxyalkyloxy, aminoalkoxy, alkylaminoalkoxy, dialkylaminoalkoxy, carboxy, and cyano. Within the above optional substituents on “cycloalkyl”, the alkyl and alkenyl, either alone or as part of another substituent on the cycloalkyl ring, are independently optionally substituted with one, two, three, four, or five halo, e.g. haloalkyl, haloalkoxy, haloalkenyloxy, or haloalkylsulfonyl.

“Optionally substituted cycloalkylalkyl” means an alkyl group substituted with at least one, specifically one or two, optionally substituted cycloalkyl groups, as defined herein.

“Optionally substituted heteroaryl” means a heteroaryl group optionally substituted with one, two, or three substituents independently selected from acyl, acylamino, acyloxy, optionally substituted alkyl, optionally substituted alkenyl, alkoxy, alkenyloxy, halo, hydroxy, alkoxycarbonyl, alkenyloxycarbonyl, amino, alkylamino, dialkylamino, nitro, aminocarbonyl, alkylaminocarbonyl, dialkylaminocarbonyl, carboxy, cyano, alkylthio, alkylsulfinyl, alkylsulfonyl, aminosulfonyl, alkylaminosulfonyl, dialkylaminosulfonyl, alkylsulfonylamino, aminoalkoxy, alkylaminoalkoxy, and dialkylaminoalkoxy. Within the optional substituents on “heteroaryl”, the alkyl and alkenyl, either alone or as part of another group (including, for example, the alkyl in alkoxycarbonyl), are independently optionally substituted with one, two, three, four, or five halo.

“Optionally substituted heteroarylalkyl” means an alkyl group, as defined herein, substituted with at least one, specifically one or two, optionally substituted heteroaryl group(s), as defined herein.

“Optionally substituted heterocycloalkyl” means a heterocycloalkyl group, as defined herein, optionally substituted with one, two, or three substituents independently selected from acyl, acylamino, acyloxy, optionally substituted alkyl, optionally substituted alkenyl, alkoxy, alkenyloxy, halo, hydroxy, alkoxycarbonyl, alkenyloxycarbonyl, amino, alkylamino, aminocarbonyl, alkylaminocarbonyl, dialkylaminocarbonyl, carboxy, cyano, alkylthio, alkylsulfinyl, alkylsulfonyl, aminosulfonyl, alkylaminosulfonyl, dialkylaminosulfonyl, alkylsulfonylamino, aminoalkoxy, or aryl is pentafluorophenyl. Within the optional substituents on “heterocycloalkyl”, the alkyl and alkenyl, either alone or as part of another group (including, for example, the alkyl in alkoxycarbonyl), are independently optionally substituted with one, two, three, four, or five halo.

“Optionally substituted heterocycloalkylalkyl” means an alkyl group, as defined herein, substituted with at least one, specifically one or two, optionally substituted heterocycloalkyl group(s) as defined herein.

“Hematologic malignancies” are types of cancers that affect blood, bone marrow, and lymph nodes. Hematologic malignancies include, but are not limited to non-Hodgkin lymphoma (NHL) including aggressive B-cell lymphomas (Diffuse Large B-cell Lymphoma, Mantle Cell Lymphoma and Burkitt's Lymphoma), and indolent B-cell lymphomas (Follicular Lymphoma, Marginal Zone Lymphomas; Chronic Lymphocytic Leukemia/Small Lymphocytic Lymphoma, and Lymphoplasmacytic Lymphoma; mantle cell lymphoma (MCL), diffuse large B-cell lymphoma (DLBCL); Marginal Zone Lymphomas (MZLs) including extranodal MZL (MALT lymphoma), nodal MZL and splenic MZL (NCCN, 2010); and Lymphoplasmacytic lymphoma, also termed Waldenstrom's magroglobulinemia.

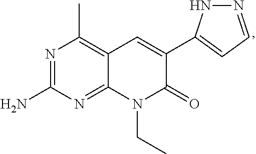 [SMILES: CCn1c(=O)c(-c2ccn[nH]2)cc2c(C)nc(N)nc21]

As used herein, “Compound A” means the structure known by its name 2-amino-8-ethyl-4-methyl-6-(1H-pyrazol-5-yl)pyrido[2,3-d]pyrimidin-7(8H)-one. Compound A is disclosed in WO 07/044813, the entire contents of which is incorporated herein by reference.

“Bendamustine” (CAS No. 16506-27-7) means the compound known by its chemical name 4-[5-[Bis(2-chloroethyl)amino]-1-methylbenzimidazol-2-yl]butanoic acid and by its trade names RIBOMUSTIN and TREANDA, for the treatment of chronic lymphocytic leukemias and lymphomas.

“Rituximab” means the chimeric monoclonal antibody sold under the trade names RITUXAN and MABTHERA, for the treatment of lymphomas, leukemias, as well as some autoimmune disorders and transplant rejection.

“Pharmaceutical composition” comprises 1) a Compound of Formula I or a single isomer thereof where the compound is optionally as a pharmaceutically acceptable salt and additionally optionally as a hydrate and additionally optionally as a solvate thereof; 2) a pharmaceutically acceptable carrier, excipient, or diluent, and 3) optionally one or both of bendamustine and rituximab as described herein.

“Yield” for each of the reactions described herein is expressed as a percentage of the theoretical yield.

“Patient” for the purposes of the present invention includes humans and other animals, particularly mammals, and other organisms. Thus the methods are applicable to both human therapy and veterinary applications. In a preferred embodiment the patient is a mammal, and in a most preferred embodiment the patient is human.

The terms “effective amount” or “pharmaceutically effective amount” or “therapeutically effective amount” refer to a sufficient amount of an agent to provide the desired biological, therapeutic, and/or prophylactic result. That result can be reduction, amelioration, palliation, lessening, delaying, and/or alleviation of one or more of the signs, symptoms, or causes of a disease, or any other desired alteration of a biological system. In reference to cancer, an effective amount comprises an amount sufficient to cause a tumor to shrink and/or to decrease the growth rate of the tumor (such as to suppress tumor growth) or to prevent or delay other unwanted cell proliferation. In some embodiments, an effective amount is an amount sufficient to delay development. In some embodiments, an effective amount is an amount sufficient to prevent or delay recurrence. An effective amount can be administered in one or more administrations. The effective amount of the drug or composition may: (i) reduce the number of cancer cells; (ii) reduce tumor size; (iii) inhibit, retard, slow to some extent and preferably stop cancer cell infiltration into peripheral organs; (iv) inhibit (i.e., slow to some extent and preferably stop) tumor metastasis; (v) inhibit tumor growth; (vi) prevent or delay occurrence and/or recurrence of tumor; and/or (vii) relieve to some extent one or more of the symptoms associated with the cancer. For example, an “effective amount” for therapeutic uses is the amount of Compound A or a metabolite thereof or a pharmaceutically acceptable salt or solvate thereof, or a composition comprising Compound A or a metabolite thereof or a pharmaceutically acceptable salt thereof, required to provide a clinically significant decrease in relapsed or refractory indolent B-cell Non-Hodgkin Lymphoma, Mantle Cell Lymphoma or Chronic Lymphocytic Leukemia or a slowing of progression of the refractory indolent B-cell Non-Hodgkin Lymphoma, Mantle Cell Lymphoma or Chronic Lymphocytic Leukemia.

A “pharmaceutically acceptable salt” ala compound means a salt that is pharmaceutically acceptable and that possesses the desired pharmacological activity of the parent compound. It is understood that the pharmaceutically acceptable salts are non-toxic. Additional information on suitable pharmaceutically acceptable salts can be found in Remington's Pharmaceutical Sciences, 17^{th} ed., Mack Publishing Company, Easton, Pa., 1985, which is incorporated herein by reference or S. M. Berge, et al., “Pharmaceutical Salts,” J. Pharm. Sci., 1977; 66:1-19 both of which are incorporated herein by reference.

Examples of pharmaceutically acceptable acid addition salts include those formed with inorganic acids such as hydrochloric acid, hydrobromic acid, sulfuric acid, nitric acid, phosphoric acid, and the like; as well as organic acids such as acetic acid, trifluoroacetic acid, propionic acid, hexanoic acid, cyclopentanepropionic acid, glycolic acid, pyruvic acid, lactic acid, oxalic acid, maleic acid, malonic acid, succinic acid, fumaric acid, tartaric acid, citric acid, benzoic acid, cinnamic acid, 3-(4-hydroxybenzoyl)benzoic acid, mandelic acid, methanesulfonic acid, ethanesulfonic acid, 1,2-ethanedisulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, p-toluenesulfonic acid, and salicylic acid and the like.

Examples of a pharmaceutically acceptable base addition salts include those formed when an acidic proton present in the parent compound is replaced by a metal ion, such as sodium, potassium, lithium, ammonium, calcium, magnesium, iron, zinc, copper, manganese, aluminum salts and the like. Preferable salts are the ammonium, potassium, sodium, calcium, and magnesium salts. Salts derived from pharmaceutically acceptable organic non-toxic bases include, but are not limited to, salts of primary, secondary, and tertiary amines, substituted amines including naturally occurring substituted amines, cyclic amines and basic ion exchange resins. Examples of organic bases include isopropylamine, trimethylamine, diethylamine, triethylamine, tripropylamine, ethanolamine, 2-dimethylaminoethanol, 2-diethylaminoethanol, dicyclohexylamine, lysine, arginine, histidine, caffeine, procaine, hydrabamine, choline, betaine, ethylenediamine, glucosamine, methylglucamine, theobromine, purines, piperazine, piperidine. N-ethylpiperidine, tromethamine, N-methylglucamine, polyamine resins, and the like. Exemplary organic bases are isopropylamine, diethylamine, ethanolamine, trimethylamine, dicyclohexylamine, choline, and caffeine.

“Prodrug” refers to compounds that are transformed (typically rapidly) in vivo to yield the parent compound of the above formulae, for example, by hydrolysis in blood. Common examples include, but are not limited to, ester and amide forms of a compound having an active form bearing a carboxylic acid moiety. Examples of pharmaceutically acceptable esters of the compounds of this invention include, but are not limited to, alkyl esters (for example with between about one and about six carbons) the alkyl group is a straight or branched chain. Acceptable esters also include cycloalkyl esters and arylalkyl esters such as, but not limited to benzyl. Examples of pharmaceutically acceptable amides of the compounds of this invention include, but are not limited to, primary amides, and secondary and tertiary alkyl amides (for example with between about one and about six carbons). Amides and esters of the compounds of the present invention may be prepared according to conventional methods. A thorough discussion of prodrugs is provided in T. Higuchi and V. Stella, “Pro-drugs as Novel Delivery Systems,” Vol 14 of the A.C.S. Symposium Series, and in Bioreversible Carriers in Drug Design, ed. Edward B. Roche, American Pharmaceutical Association and Pergamon Press, 1987, both of which are incorporated herein by reference for all purposes.

“Metabolite” refers to the break-down or end product of a compound or its salt produced by metabolism or biotransformation in the animal or human body; for example, biotransformation to a more, polar molecule such as by oxidation, reduction, or hydrolysis, or to a conjugate (see Goodman and Gilman, “The Pharmacological Basis of Therapeutics” 8.sup.th Ed., Pergamon Press. Gilman et al. (eds), 1990 for a discussion of biotransformation). As used herein, the metabolite of a compound of the invention or its salt may be the biologically active form of the compound in the body. In one example, a prodrug may be used such that the biologically active form, a metabolite, is released in vivo. In another example, a biologically active metabolite is discovered serendipitously, that is, no prodrug design per se was undertaken. An assay for activity of a metabolite of a compound of the present invention is known to one of skill in the art in light of the present disclosure.

Unless otherwise indicated. “treating” or “treatment” of a disease, disorder, or syndrome, as used herein, means inhibiting the disease, disorder, or syndrome, that is, arresting its development; and relieving the disease, disorder, or syndrome, that is, causing regression of the disease, disorder, or syndrome. As is known in the art, in the context of treatment, adjustments for systemic versus localized delivery, age, body weight, general health, sex, diet, time of administration, drug interaction and the severity of the condition may be necessary, and will be ascertainable with routine experimentation by one of ordinary skill in the art.

“Prevention” means preventing the disease, disorder, or syndrome from occurring in a human, i.e. causing the clinical symptoms of the disease, disorder, or syndrome not to develop in an animal that may be exposed to or predisposed to the disease, disorder, or syndrome but does not yet experience or display symptoms of the disease, disorder, or syndrome.

### Embodiments

The following paragraphs present a number of embodiments that can be used to practice the invention. In each instance, the embodiment includes both the recited compounds as well as individual isomers and mixtures of isomers. In addition, in each instance, the embodiment includes the pharmaceutically acceptable salts, hydrates, and/or solvates of the recited compounds and any individual isomers or mixture of isomers thereof.

In one embodiment, methods are provided for treating cancer comprising administering to a patient an effective amount of a Compound of Formula I or a pharmaceutical composition comprising a Compound of Formula I in combination with one or both of bendamustine and rituximab.

In another embodiment, methods are provided for treating cancer comprising administering to a patient an effective amount of a Compound of Formula I or a pharmaceutical composition comprising a Compound of Formula I in combination with one or both of bendamustine and rituximab where the cancer is a hematologic malignancy. In some embodiments, the hematologic malignancy is non-Hodgkin lymphoma (NHL), B-cell lymphomas, including Diffuse Large B-call Lymphoma (DLBCL), Mantle Cell Lymphoma (MCL), Burkitt's Lymphoma, Follicular Lymphoma (FL), Marginal Zone Lymphomas (MZL), Chronic Lymphocytic Leukemia (CLL)/Small Lymphocytic Lymphoma, and Lymphoplasmacytic Lymphoma. In other embodiments, the hematologic malignancy is relapsed or refractory indolent′ B-cell Non-Hodgkin Lymphoma or Chronic Lymphocytic Leukemia.

Any of the following embodiments, including the representative compounds described below, may be used to practice any of the methods disclosed herein.

### Compounds of Formula I

In one embodiment of the Compound of Formula I used in the method, R¹ is hydrogen, optionally substituted alkyl, optionally substituted cycloalkyl, optionally substituted cycloalkylalkyl, optionally substituted aryl, optionally substituted arylalkyl, optionally substituted heterocycloalkyl, optionally substituted heterocycloalkylalkyl, optionally substituted heteroaryl or optionally substituted heteroarylalkyl. Specifically, R¹ is hydrogen, optionally substituted alkyl, optionally substituted cycloalkyl, optionally substituted arylalkyl, or optionally substituted heterocycloalkylalkyl. More specifically, R¹ is hydrogen, alkyl, alkyl substituted with one or two hydroxy, alkyl substituted with alkoxy, cycloalkyl, arylalkyl, or heterocycloalkylalkyl. Even more specifically, R¹ is hydrogen, methyl, ethyl, propyl, isopropyl, 2-hydroxypropyl, 3-hydroxypropyl, 2-ethoxyethyl, 3-methoxypropyl, 3-ethoxypropyl, 3-isopropoxypropyl, cyclopropyl, cyclobutyl, cyclopentyl, cyclohexyl, benzyl, or 2-piperidin-1-ylethyl. Yet even more specifically, R¹ is ethyl, isopropyl, cyclopentyl, or cyclohexyl. Yet even more specifically, R¹ is ethyl.

In another embodiment of the Compound of Formula I used in the method, R² is hydrogen or alkyl where the alkyl is optionally substituted with 1, 2, 3, 4, or 5 R⁸ groups. Specifically, R² is hydrogen or alkyl where the alkyl is optionally substituted with one, two, or three R⁸ groups. More specifically, R² is hydrogen or alkyl where the alkyl is optionally substituted with one, two, or three R⁸ groups; and each R⁸, when present, is independently selected from amino, alkylamino, dialkylamino, and halo. Even more specifically, R² is hydrogen, methyl, ethyl, propyl, isopropyl, tert-butyl, 3-aminopropyl, 3-(N-methylamino)-propyl, 3-(N,N-dimethylamino)-propyl, 2-fluoroethyl, or 2,2,2-trifluoroethyl. Yet even more specifically, R² is hydrogen or ethyl. Yet even more preferably, R² is hydrogen.

In another embodiment, R² is hydrogen.

In another embodiment, R² is alkyl optionally substituted with 1, 2, 3, 4, or 5, R⁸ groups. Specifically, R² is alkyl where the alkyl is optionally substituted with one, two, or three R⁸ groups; and each R⁸, when present, is independently selected from amino, alkylamino, dialkylamino, and halo. Even more specifically, R² is methyl, ethyl, propyl, isopropyl, tert-butyl, 3-aminopropyl, 3-(N-methylamino)-propyl, 3-(N,N-dimethylamino)-propyl, 2-fluoroethyl, or 2,2,2-trifluoroethyl. Yet even more specifically, R² is ethyl.

In another embodiment, R⁴ is optionally substituted alkyl. Specifically, R⁴ is methyl or ethyl. More specifically, R is methyl.

In another embodiment, R⁶ is acyl. More specifically, R⁶ is alkylcarbonyl. Even more specifically, R⁶ is acetyl.

In another embodiment, R⁶ is phenyl optionally substituted with 1, 2, 3, 4, or 5 R⁹ groups. Specifically, R⁶ is phenyl optionally substituted with one or two R⁹ groups; and each R⁹, when present, is independently selected from aryl, halo, alkoxy, aryloxy, and haloalkyl. More specifically, R⁶ is phenyl optionally substituted with one or two R⁹ groups; and each R⁹, when present, is independently selected from phenyl, fluoro, chloro, methoxy, phenyloxy, and trifluoromethyl. Even more specifically, R⁶ is phenyl, phenyl substituted with phenyl, fluorophenyl, difluorophenyl, chlorophenyl, dichlorophenyl, phenyl substituted with chloro and fluoro, methoxyphenyl, dimethoxyphenyl, phenyloxyphenyl, or trifluoromethylphenyl. Yet even more specifically, R⁶ is phenyl, 2-phenyl-phenyl, 3-phenyl-phenyl, 4-phenyl-phenyl, 2-fluorophenyl, 3-fluorophenyl, 4-fluorophenyl, 2,3-di fluorophenyl, 2,4-difluorophenyl, 2,5-difluorophenyl, 2,6-difluorophenyl, 3,4-difluorophenyl, 3,5-difluorophenyl, 2-chlorophenyl, 3-chlorophenyl, 4-chlorophenyl, 2,3-dichlorophenyl, 2,4-dichlorophenyl, 2,5-dichlorophenyl, 2,6-dichlorophenyl, 3,4-dichlorophenyl, 3,5-dichlorophenyl, 3-chloro-4-fluoro-phenyl, 2-methoxyphenyl, 3-methoxyphenyl, 4-methoxyphenyl, 2,3-dimethoxyphenyl, 2,4-dimethoxyphenyl, 2,5-dimethoxyphenyl, 2,6-dimethoxyphenyl, 3,4-dimethoxyphenyl, 3,5-dimethoxyphenyl, 4-phenyloxyphenyl, 2-tri fluoromethylphenyl, 3-trifluoromethylphenyl, or 4-trifluoromethylphenyl.

In another embodiment, R⁶ is phenyl substituted with 1, 2, 3, 4, or 5 R⁹ groups.

In another embodiment. R⁶ is heteroaryl optionally substituted with 1, 2, 3, 4, or 5 R⁹ groups.

In another embodiment, R⁶ is a 6-membered heteroaryl optionally substituted with one or two R⁹. More specifically, R⁶ is pyridinyl, pyrazinyl, pyrimidinyl, or pyridazinyl each of which is optionally substituted with one R⁹ where R⁹, when present, is halo. Even more specifically, R⁶ is pyridiN-2-yl, pyridiN-3-yl, pyridiN-4-yl, 3-fluoropyridiN-4-yl, pyrazin-2-yl, pyrazin-3-yl, pyrimidin-2-yl, pyrimidin-4-yl, pyrimidin-5-yl, pyridazin-3-yl, or pyridazin-4-yl, each of which is optionally substituted with one or two R⁹.

In another embodiment, R⁶ is pyrazinyl, pyrimidinyl, or pyridazinyl each of which is optionally substituted with one R⁹ where R⁹, when present, is halo. Even more specifically, R⁶ is pyrazin-2-yl, pyrazin-3-yl, pyrimidin-2-yl, pyrimidin-4-yl, pyrimidin-5-yl, pyridazin-3-yl, or pyridazin-4-yl.

In another embodiment. R⁶ is 5-membered heteroaryl optionally substituted with one or two R⁹. Specifically R⁶ is pyrazolyl, imidazolyl, thienyl, thiazolyl, oxazolyl, isoxazolyl, oxadiazolyl, furanyl, pyrrolyl, triazolyl, or tetrazolyl, each of which is optionally substituted with one R⁹ where R⁹, when present, is alkyl, arylalkyl, cyano, aryl, alkoxycarbonyl, or halo. More specifically, R⁶ is pyrazol-1-yl, pyrazol-3-yl, pyrazol-4-yl, pyrazol-5-yl, imidazol-1-yl, imidazol-2-yl, imidazol-5-yl, thien-2-yl, thien-3-yl, thiazol-2-yl, thiazol-4-yl, thiazol-5-yl, oxazol-2-yl, oxazol-4-yl, oxazol-5-yl, isoxazol-3-yl, isoxazol-4-yl, isoxazol-5-yl, 1,2,3-oxadiazol-4-yl, 1,2,3-oxadiazol-5-yl, 1,3,4-oxadiazol-2-yl, 1,2,4-oxadiazol-5-yl, furan-2-yl, furan-3-yl, pyrrol-1-yl, pyrrol-2-yl, pyrrol-3-yl, triazol-1-yl, triazol-4-yl, triazol-5-yl, tetrazol-1-yl, or tetrazol-5-yl; each of which is optionally substituted with one R⁹ where R⁹, when present, is methyl, benzyl, cyano, phenyl, N-tert-butoxycarbonyl, or chloro. Even more specifically, R⁶ is pyrazol-3-yl, pyrazol-4-yl, pyrazol-5-yl, imidazol-2-yl, imidazol-4-yl, imidazol-5-yl, thien-2-yl, thien-3-yl, thiazol-2-yl, thiazol-4-yl, thiazol-5-yl, oxazol-2-yl, oxazol-4-yl, oxazol-5-yl, isoxazol-3-yl, isoxazol-4-yl, isoxazol-5-yl, 1,2,3-oxadiazol-5-yl, 1,3,4-oxadiazol-2-yl, 1,2,4-oxadiazol-3-yl, 1,2,4-oxadiazol-5-yl, furan-2-yl, furan-3-yl, pyrrol-2-yl, pyrrol-3-yl, triazol-4-yl, triazol-5-yl, or tetrazol-5-yl; each of which is optionally substituted with one R⁹ where R⁹, when present, is methyl, benzyl, cyano, phenyl, N-tert-butoxycarbonyl, or chloro.

In another embodiment, R⁶ is thienyl, pyrrolyl, furanyl, pyrazolyl, thiazolyl, isoxazolyl, imidazolyl, triazolyl, or tetrazolyl, each of which is optionally substituted with one R⁹ where R⁹, when present, is methyl, benzyl, cyano, phenyl, N-tert-butoxycarbonyl, or chloro. Specifically, R⁶ is thien-2-yl, thien-3-yl, pyrrol-2-yl, furan-2-yl, furan-3-yl, pyrazol-3-yl, pyrazol-4-yl, pyrazol-5-yl, thiazol-2-yl, thiazol-5-yl, isoxazol-4-yl, imidazol-5-yl, triazol-5-yl, tetrazol-5-yl, each of which is optionally substituted with one R⁹ where R⁹, when present, is methyl, benzyl, cyano, phenyl. N-tert-butoxycarbonyl, or chloro. More specifically, R⁶ is thien-2-yl, thien-3-yl, 5-cyano-thien-2-yl, 4-methyl-thien-2-yl, 4-methyl-thien-3-yl, 5-chloro-thien-5-yl, 5-phenyl-thien-2-yl, pyrrol-2-yl, N-tert-butoxycarbonyl-pyrrol-2-yl, N-methyl-pyrrol-2-yl, furan-2-yl, furan-3-yl, pyrazol-3-yl, pyrazol-4-yl, N-benzyl-pyrazol-4-yl, pyrazol-5-yl, thiazol-2-yl, thiazol-5-yl, isoxazol-4-yl, imidazol-5-yl, triazol-5-yl, tetrazol-5-yl,

In another embodiment, R⁶ is thien-2-yl, thien-3-yl, pyrrol-2-yl, furan-2-yl, furan-3-yl, pyrazol-3-yl, pyrazol-4-yl, pyrazol-5-yl, thiazol-2-yl, thiazol-5-yl, isoxazol-4-yl, imidazol-5-yl, triazol-5-yl, or tetrazol-5-yl, each of which is optionally substituted with one R⁹ where R⁹, when present, is methyl, benzyl, cyano, phenyl, N-tert-butoxycarbonyl, or chloro.

In another embodiment, R⁶ is indolyl, benzimidazolyl, benzofuranyl, benzoxazolyl, or benzoisoxazolyl each of which is optionally substituted with 1, 2, 3, 4, or 5 R⁹ groups. Specifically, R⁶ is indol-2-yl, indol-3-yl, indol-4-yl, indol-5-yl, indol-6-yl, indol-7-yl, benzimidazol-2-yl, benzimidazol-4-yl, benzimidazol-5-yl, benzimidazol-6-yl, benzimidazol-7-yl, benzofuran-2-yl, benzofuran-3-yl, benzofuran-4-yl, benzofuran-5-yl, benzofuran-6-yl, benzofuran-7-yl, benzoxazol-2-yl, benzoxazol-4-yl, benzoxazol-5-yl, benzoxazol-6-yl, benzoxazol-7-yl, benzoisoxazol-3-yl, benzoisoxazol-4-yl, benzoisoxazol-5-yl, benzoisoxazol-6-yl, or benzoisoxazol-7-yl; each of which is optionally substituted with 1, 2, 3, 4, or 5 R⁹ groups. More specifically. R⁶ is indol-6-yl.

In another embodiment, R¹ is hydrogen, optionally substituted alkyl, optionally substituted cycloalkyl, optionally substituted heterocycloalkylalkyl, or optionally substituted arylalkyl; X is —NH—; R² is hydrogen or alkyl where the alkyl is optionally substituted with one or two R⁸ groups; R⁴ is alkyl; R⁵ is hydrogen; R⁶ is phenyl or heteroaryl wherein the phenyl and heteroaryl are optionally substituted with one, two, or three R⁹ groups; each R⁸, when present, is independently amino, alkylamino, dialkylamino, or halo; and each R⁹, when present, is independently alkyl, arylalkyl, cyano, aryl, alkoxycarbonyl, or halo.

In another embodiment, R⁶ is pyrazol-3-yl, pyrazol-4-yl, pyrazol-5-yl, imidazol-2-yl, imidazol-4-yl, imidazol-5-yl, thien-2-yl, thien-3-yl, thiazol-2-yl, thiazol-4-yl, thiazol-5-yl, oxazol-2-yl, oxazol-4-yl, isoxazol-3-yl, isoxazol-4-yl, isoxazol-5-yl, 1,2,3-oxadiazol-4-yl, 1,2,3-oxadiazol-5-yl, 1,3,4-oxadiazol-2-yl, 1,2,4-oxadiazol-3-yl, 1,2,4-oxadiazol-5-yl, furan-2-yl, furan-3-yl, pyrrol-2-yl, pyrrol-3-yl, triazol-4-yl, triazol-5-yl, or tetrazol-5-yl; each of which is optionally substituted with 1, 2, 3, 4, or 5 R⁹ groups.

In another embodiment, R¹ is alkyl or cycloalkyl; R⁴ is methyl; and R⁶ is heteroaryl optionally substituted with one or two R⁹ groups. Specifically, each R⁹, when present, is independently alkyl, arylalkyl, cyano, aryl, alkoxycarbonyl, or halo. Specifically, R⁶ is pyrazol-3-yl, pyrazol-4-yl, pyrazol-5-yl, imidazol-2-yl, imidazol-4-yl, imidazol-5-yl, thien-2-yl, thien-3-yl, thiazol-2-yl, thiazol-4-yl, thiazol-5-yl, oxazol-2-yl, oxazol-4-yl, oxazol-5-yl, isoxazol-3-yl, isoxazol-4-yl, isoxazol-5-yl, 1,2,3-oxadiazol-4-yl, 1,2,3-oxadiazol-5-yl, 1,3,4-oxadiazol-2-yl, 1,2,4-oxadiazol-3-yl, 1,2,4-oxadiazol-5-yl, furan-2-yl, furan-3-yl, pyrrol-2-yl, pyrrol-3-yl, triazol-4-yl, triazol-5-yl, or tetrazol-5-yl; each of which is optionally substituted with one R⁹ where R⁹, when present, is methyl, benzyl, cyano, phenyl, or N-tert-butoxycarbonyl.

In another embodiment, R² is hydrogen.

In another embodiment, R² is methyl or ethyl.

In another embodiment, R¹ is alkyl or cycloalkyl; R⁴ is methyl; and R⁶ is phenyl optionally substituted with one or two R⁹ groups. Specifically each R⁹, when present, is independently halo, alkoxy, or haloalkyl.

In another embodiment, R¹ is alkyl or cycloalkyl; R⁴ is methyl; and R² is hydrogen.

In another embodiment, R¹ is alkyl or cycloalkyl; R⁴ is methyl; and R² is optionally substituted alkyl.

Representative compounds of Formula I are depicted below. The examples are merely illustrative and do not limit the scope of the invention in any way. Compounds of the invention are named according to systematic application of the nomenclature rules agreed upon by the International Union of Pure and Applied Chemistry (IUPAC), International Union of Biochemistry and Molecular Biology (IUBMB), and the Chemical Abstracts Service (CAS). Names were generated using ACD/Labs naming software 8.00 release, product version 8.08.

TABLE I

| Example | Structure | Name |
|---|---|---|
| 1 | 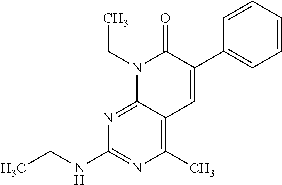 [SMILES: CCNc1nc(C)c2cc(-c3ccccc3)c(=O)n(CC)c2n1] | 8-ethyl-2-(ethylamino)-4-methyl-6- phenylpyrido[2,3-d]pyrimidin- 7(8H)-one |
| 2 | 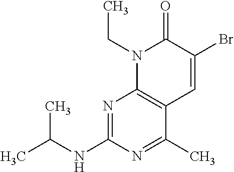 [SMILES: CCn1c(=O)c(Br)cc2c(C)nc(NC(C)C)nc21] | 6-bromo-8-ethyl-4-methyl-2-[(1- methylethyl)amino]pyrido[2,3- d]pyrimidin-7(8H)-one |
| 3 | 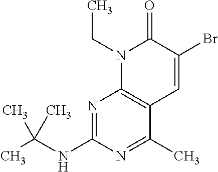 [SMILES: CCn1c(=O)c(Br)cc2c(C)nc(NC(C)(C)C)nc21] | 6-bromo-2-[(1,1- dimethylethyl)amino]-8-ethyl-4- methylpyrido[2,3-d]pyrimidin- 7(8H)-one |
| 4 | 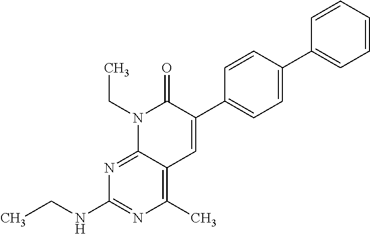 [SMILES: CCNc1nc(C)c2cc(-c3ccc(-c4ccccc4)cc3)c(=O)n(CC)c2n1] | 6-biphenyl-4-yl-8-ethyl-2- (ethylamino)-4-methylpyrido[2,3- d]pyrimidin-7(8H)-one |
| 5 | 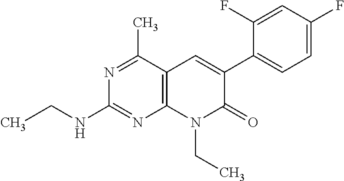 [SMILES: CCNc1nc(C)c2cc(-c3ccc(F)cc3F)c(=O)n(CC)c2n1] | 6-(2,4-difluorophenyl)-8-ethyl-2- (ethylamino)-4-methylpyrido[2,3- d]pyrimidin-7(8H)-one |
| 6 | 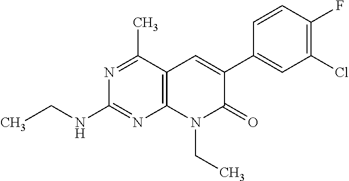 [SMILES: CCNc1nc(C)c2cc(-c3ccc(F)c(Cl)c3)c(=O)n(CC)c2n1] | 6-(3-chloro-4-fluorophenyl)-8- ethyl-2-(ethylamino)-4- methylpyrido[2,3-d]pyrimidin- 7(8H)-one |
| 7 | 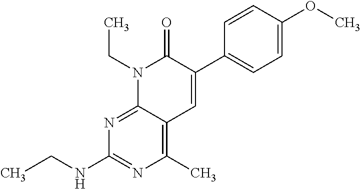 [SMILES: CCNc1nc(C)c2cc(-c3ccc(OC)cc3)c(=O)n(CC)c2n1] | 8-ethyl-2-(ethylamino)-4-methyl-6- [4-(methyloxy)phenyl]pyrido[2,3- d]pyrimidin-7(8H)-one |
| 8 | 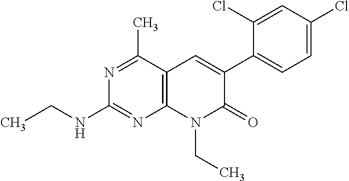 [SMILES: CCNc1nc(C)c2cc(-c3ccc(Cl)cc3Cl)c(=O)n(CC)c2n1] | 6-(2,4-dichlorophenyl)-8-ethyl-2- (ethylamino)-4-methylpyrido[2,3- d]pyrimidin-7(8H)-one |
| 9 | 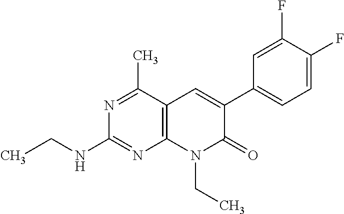 [SMILES: CCNc1nc(C)c2cc(-c3ccc(F)c(F)c3)c(=O)n(CC)c2n1] | 6-(3,4-difluorophenyl)-8-ethyl-2- (ethylamino)-4-methylpyrido[2,3- d]pyrimidin-7(8H)-one |
| 10 | 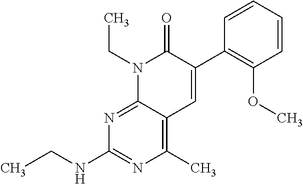 [SMILES: CCNc1nc(C)c2cc(-c3ccccc3OC)c(=O)n(CC)c2n1] | 8-ethyl-2-(ethylamino)-4-methyl-6- [2-(methyloxy)phenyl]pyrido[2,3- d]pyrimidin-7(8H)-one |
| 11 | 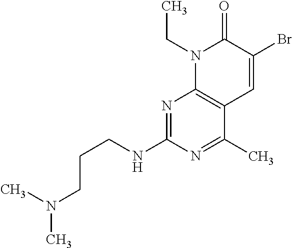 [SMILES: CCn1c(=O)c(Br)cc2c(C)nc(NCCCN(C)C)nc21] | 6-bromo-2-{[3- (dimethylamino)propyl]amino}-8- ethyl-4-methylpyrido[2,3- d]pyrimidin-7(8H)-one |
| 12 | 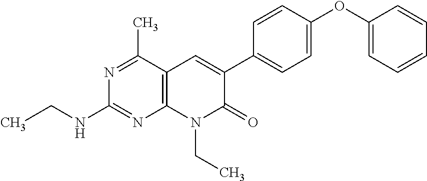 [SMILES: CCNc1nc(C)c2cc(-c3ccc(Oc4ccccc4)cc3)c(=O)n(CC)c2n1] | 8-ethyl-2-(ethylamino)-4-methyl-6- [4-(phenyloxy)phenyl]pyrido[2,3- d]pyrimidin-7(8H)-one |
| 13 | 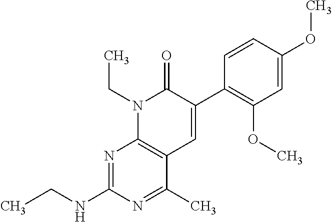 [SMILES: CCNc1nc(C)c2cc(-c3ccc(OC)cc3OC)c(=O)n(CC)c2n1] | 6-[2,4-bis(methyloxy)phenyl]-8- ethyl-2-(ethylamino)-4- methylpyrido[2,3-d]pyrimidin- 7(8H)-one |
| 14 | 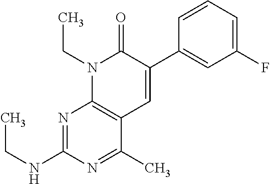 [SMILES: CCNc1nc(C)c2cc(-c3cccc(F)c3)c(=O)n(CC)c2n1] | 8-ethyl-2-(ethylamino)-6-(3- fluorophenyl)-4-methylpyrido[2,3- d]pyrimidin-7(8H)-one |
| 15 | 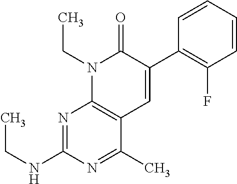 [SMILES: CCNc1nc(C)c2cc(-c3ccccc3F)c(=O)n(CC)c2n1] | 8-ethyl-2-(ethylamino)-6-(2- fluorophenyl)-4-methylpyrido[2,3- d]pyrimidin-7(8H)-one |
| 16 | 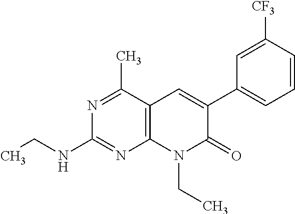 [SMILES: CCNc1nc(C)c2cc(-c3cccc(C(F)(F)F)c3)c(=O)n(CC)c2n1] | 8-ethyl-2-(ethylamino)-4-methyl- 6-[3-(trifluoromethyl)phenyl] pyrido[2,3-d]pyrimidin-7(8H)-one |
| 17 | 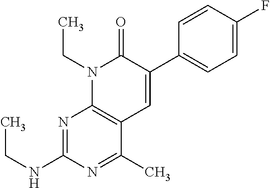 [SMILES: CCNc1nc(C)c2cc(-c3ccc(F)cc3)c(=O)n(CC)c2n1] | 8-ethyl-2-(ethylamino)-6-(4- fluorophenyl)-4-methylpyrido[2,3- d]pyrimidin-7(8H)-one |
| 18 | 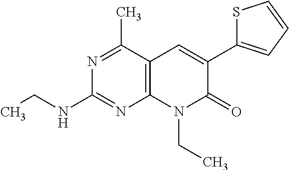 [SMILES: CCNc1nc(C)c2cc(-c3cccs3)c(=O)n(CC)c2n1] | 8-ethyl-2-(ethylamino)-4-methyl-6- (2-thienyl)pyrido[2,3-d]pyrimidin- 7(8H)-one |
| 19 | 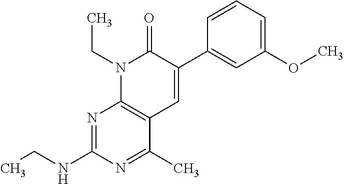 [SMILES: CCNc1nc(C)c2cc(-c3cccc(OC)c3)c(=O)n(CC)c2n1] | 8-ethyl-2-(ethylamino)-4-methyl-6- [3-(methyloxy)phenyl]pyrido[2,3- d]pyrimidin-7(8H)-one |
| 20 | 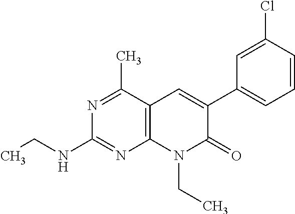 [SMILES: CCNc1nc(C)c2cc(-c3cccc(Cl)c3)c(=O)n(CC)c2n1] | 6-(3-chlorophenyl)-8-ethyl-2- (ethylamino)-4-methylpyrido[2,3- d]pyrimidin-7(8H)-one |
| 21 | 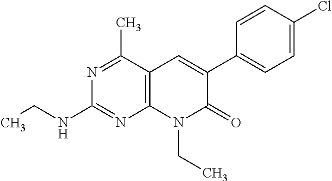 [SMILES: CCNc1nc(C)c2cc(-c3ccc(Cl)cc3)c(=O)n(CC)c2n1] | 6-(4-chlorophenyl)-8-ethyl-2- (ethylamino)-4-methylpyrido[2,3- d]pyrimidin-7(8H)-one |
| 22 | 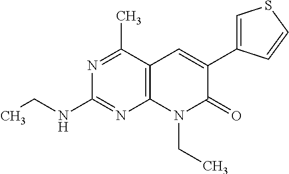 [SMILES: CCNc1nc(C)c2cc(-c3ccsc3)c(=O)n(CC)c2n1] | 8-ethyl-2-(ethylamino)-4-methyl-6- (3-thienyl)pyrido[2,3-d]pyrimidin- 7(8H)-one |
| 23 | 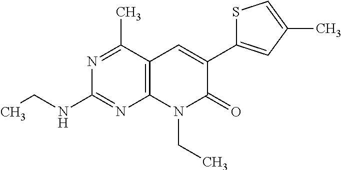 [SMILES: CCNc1nc(C)c2cc(-c3cc(C)cs3)c(=O)n(CC)c2n1] | 8-ethyl-2-(ethylamino)-4-methyl-6- (4-methyl-2-thieny)pyrido[2,3- d]pyrimidin-7(8H)-one |
| 24 | 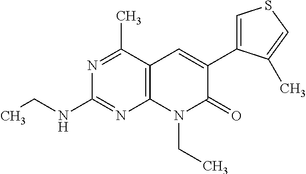 [SMILES: CCNc1nc(C)c2cc(-c3cscc3C)c(=O)n(CC)c2n1] | 8-ethyl-2-(ethylamino)-4-methyl-6- (4-methyl-3-thienyl)pyrido[2,3- d]pyrimidin-7(8H)-one |
| 25 | 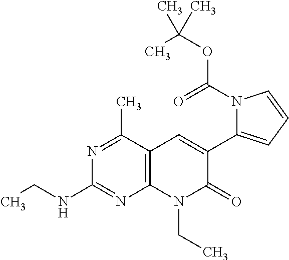 [SMILES: CCNc1nc(C)c2cc(-c3cccn3C(=O)OC(C)(C)C)c(=O)n(CC)c2n1] | 1,1-dimethylethyl 2-[8-ethyl-2- (ethylamino)-4-methyl-7-oxo-7,8- dihydropyrido[2,3-d]pyrimidin-6- yl]-1H-pyrrole-1-carboxylate |
| 26 | 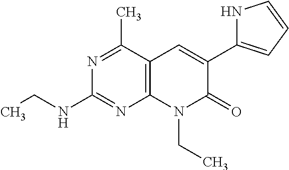 [SMILES: CCNc1nc(C)c2cc(-c3ccc[nH]3)c(=O)n(CC)c2n1] | 8-ethyl-2-(ethylamino)-4-methyl-6- (1H-pyrrol-2-yl)pyrido[2,3- d]pyrimidin-7(8H)-one |
| 27 | 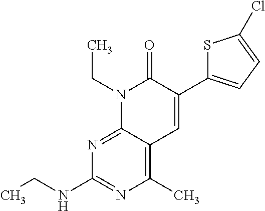 [SMILES: CCNc1nc(C)c2cc(-c3ccc(Cl)s3)c(=O)n(CC)c2n1] | 6-(5-chloro-2-thienyl)-8-ethyl-2- (ethylamino)-4-methylpyrido[2,3- d]pyrimidin-7(8H)-one |
| 28 | 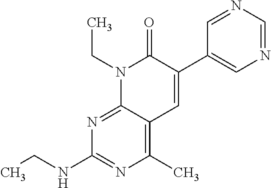 [SMILES: CCNc1nc(C)c2cc(-c3cncnc3)c(=O)n(CC)c2n1] | 8-ethyl-2-(ethylamino)-4-methyl-6- pyrimidin-5-ylpyrido[2,3- d]pyrimidin-7(8H)-one |
| 29 | 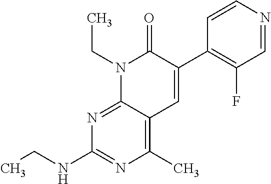 [SMILES: CCNc1nc(C)c2cc(-c3ccncc3F)c(=O)n(CC)c2n1] | 8-ethyl-2-(ethylamino)-6-(3- fluoropyridiN-4-yl)-4- methylpyrido[2,3-d]pyrimidin- 7(8H)-one |
| 30 | 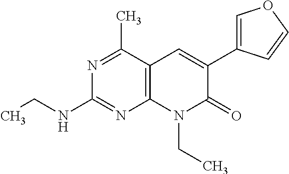 [SMILES: CCNc1nc(C)c2cc(-c3ccoc3)c(=O)n(CC)c2n1] | 8-ethyl-2-(ethylamino)-6-furan-3- yl-4-methylpyrido[2,3-d]pyrimidin- 7(8H)-one |
| 31 | 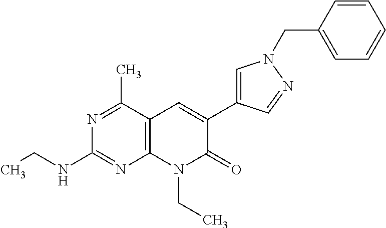 [SMILES: CCNc1nc(C)c2cc(-c3cnn(Cc4ccccc4)c3)c(=O)n(CC)c2n1] | 8-ethyl-2-(ethylamino)-4-methyl-6- [1-(phenylmethyl)-1H-pyrazol-4- yl]pyrido[2,3-d]pyrimidin-7(8H)- one |
| 32 | 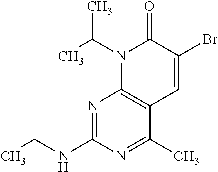 [SMILES: CCNc1nc(C)c2cc(Br)c(=O)n(C(C)C)c2n1] | 6-bromo-2-(ethylamino)-4-methyl- 8-(1-methylethyl)pyrido[2,3- d]pyrimidin-7(8H)-one |
| 33 | 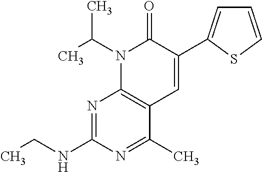 [SMILES: CCNc1nc(C)c2cc(-c3cccs3)c(=O)n(C(C)C)c2n1] | 2-(ethylamino)-4-methyl-8-(1- methylethyl)-6-(2- thienyl)pyrido[2,3-d]pyrimidin- 7(8H)-one |
| 34 | 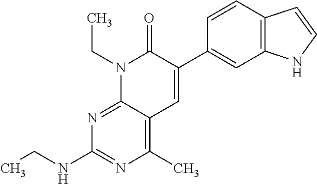 [SMILES: CCNc1nc(C)c2cc(-c3ccc4cc[nH]c4c3)c(=O)n(CC)c2n1] | 8-ethyl-2-(ethylamino)-6-(1H-indol- 6-yl)-4-methylpyrido[2,3- d]pyrimidin-7(8H)-one |
| 35 | 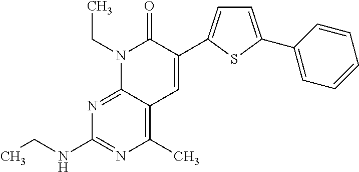 [SMILES: CCNc1nc(C)c2cc(-c3ccc(-c4ccccc4)s3)c(=O)n(CC)c2n1] | 8-ethyl-2-(ethylamino)-4-methyl-6- (5-phenyl-2-thienyl)pyrido[2,3- d]pyrimidin-7(8H)-one |
| 36 | 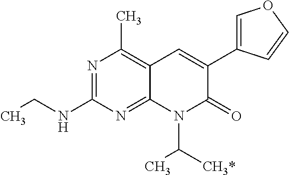 | 2-(ethylamino)-6-furan-3-yl-4- methyl-8-(1- methylethyl)pyrido[2,3- d]pyrimidin-7(8H)-one |
| 37 | 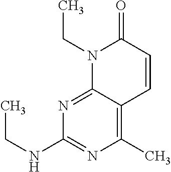 [SMILES: CCNc1nc(C)c2ccc(=O)n(CC)c2n1] | 8-ethyl-2-(ethylamino)-4- methylpyrido[2,3-d]pyrimidin- 7(8H)-one |
| 38 | 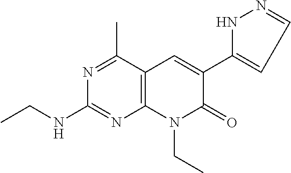 [SMILES: CCNc1nc(C)c2cc(-c3ccn[nH]3)c(=O)n(CC)c2n1] | 8-ethyl-2-(ethylamino)-4-methyl-6- (1H-pyrazol-5-yl)pyrido[2,3- d]pyrimidin-7(8H)-one |
| 39 | 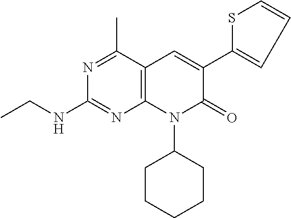 [SMILES: CCNc1nc(C)c2cc(-c3cccs3)c(=O)n(C3CCCCC3)c2n1] | 8-cyclohexyl-2-(ethylamino)-4- methyl-6-(2-thienyl)pyrido[2,3- d]pyrimidin-7(8H)-one |
| 40 | 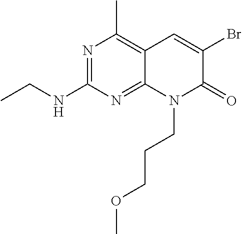 [SMILES: CCNc1nc(C)c2cc(Br)c(=O)n(CCCOC)c2n1] | 6-bromo-2-(ethylamino)-4-methyl- 8[3-(methyloxy)propyl]pyrido[2,3- d]pyrimidin-7(8H)-one |
| 41 | 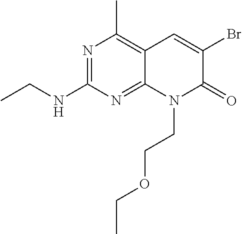 [SMILES: CCNc1nc(C)c2cc(Br)c(=O)n(CCOCC)c2n1] | 6-bromo-2-(ethylamino)-8-[2- (ethyloxy)ethyl]-4- methylpyriclo[2,3-d]pyrimidin- 7(8H)-one |
| 42 | 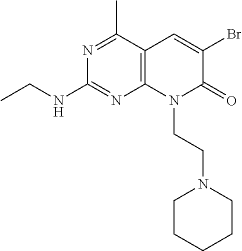 [SMILES: CCNc1nc(C)c2cc(Br)c(=O)n(CCN3CCCCC3)c2n1] | 6-bromo-2-(ethylamino)-4-methyl- 8-(2-piperidin-1-ylethyl)pyrido[2,3- d]pyrimidin-7(8H)-one |
| 43 | 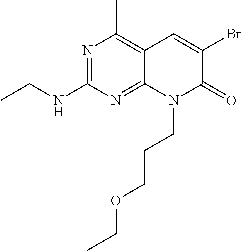 [SMILES: CCNc1nc(C)c2cc(Br)c(=O)n(CCCOCC)c2n1] | 6-bromo-2-(ethylamino)-8-[3- (ethyloxy)propyl]-4- methylpyrido[2,3-d]pyrimidin- 7(8H)-one |
| 44 | 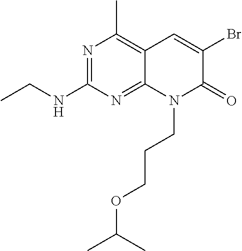 [SMILES: CCNc1nc(C)c2cc(Br)c(=O)n(CCCOC(C)C)c2n1] | 6-bromo-2-(ethylamino)-4-methyl- 8-{3-[(1-methylethyl)oxy] propyl}pyrido[2,3-d]pyrimidin- 7(8H)-one |
| 45 | 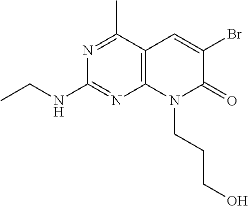 [SMILES: CCNc1nc(C)c2cc(Br)c(=O)n(CCCO)c2n1] | 6-bromo-2-(ethylamino)-8-(3- hydroxypropyl)-4- methylpyrido[2,3-d]pyrimidin- 7(8H)-one |
| 46 | 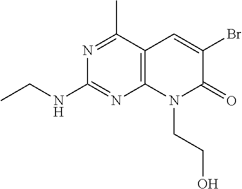 [SMILES: CCNc1nc(C)c2cc(Br)c(=O)n(CCO)c2n1] | 6-bromo-2-(ethylamino)-8-(2- hydroxyethyl)-4-methylpyrido[2,3- d]pyrimidin-7(8H)-one |
| 47 | 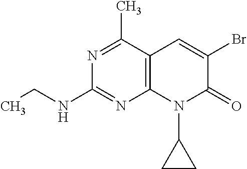 [SMILES: CCNc1nc(C)c2cc(Br)c(=O)n(C3CC3)c2n1] | 6-bromo-8-cyclopropyl-2- (ethylamino)-4-methylpyrido[2,3- d]pyrimidin-7(8H)-one |
| 48 | 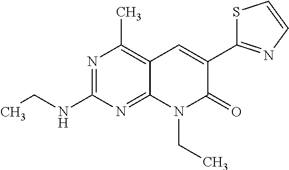 [SMILES: CCNc1nc(C)c2cc(-c3nccs3)c(=O)n(CC)c2n1] | 8-ethyl-2-(ethylamino)-4-methyl-6- (1,3-thiazol-2-yl)pyrido[2,3- d]pyrimidin-7(8H)-one |
| 49 | 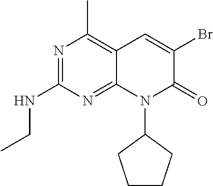 [SMILES: CCNc1nc(C)c2cc(Br)c(=O)n(C3CCCC3)c2n1] | 6-bromo-8-cyclopentyl-2- (ethylamino)-4-methylpyrido[2,3- d]pyrimidin-7(8H)-one |
| 50 | 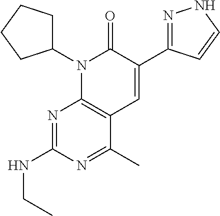 [SMILES: CCNc1nc(C)c2cc(-c3cc[nH]n3)c(=O)n(C3CCCC3)c2n1] | 8-cyclopentyl-2-(ethylamino)-4- methyl-6-(1H-pyrazol-3- yl)pyrido[2,3-d]pyrimidin-7(8H)- one |
| 51 | 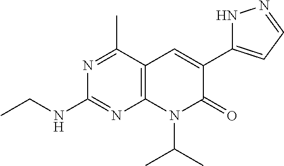 [SMILES: CCNc1nc(C)c2cc(-c3ccn[nH]3)c(=O)n(C(C)C)c2n1] | 2-(ethylamino)-4-methyl-8-(1- methylethyl)-6-(1H-pyrazol-5- yl)pyrido[2,3-d]pyrimidin-7(8H)- one |
| 52 | 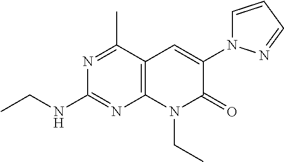 [SMILES: CCNc1nc(C)c2cc(-n3cccn3)c(=O)n(CC)c2n1] | 8-ethyl-2-(ethylamino)-4-methyl-6- (1H-pyrazol-1-yl)pyrido[2,3- d]pyrimidin-7(8H)-one |
| 53 | 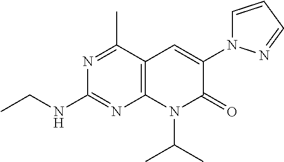 [SMILES: CCNc1nc(C)c2cc(-n3cccn3)c(=O)n(C(C)C)c2n1] | 2-(ethylamino)-4-methyl-8-(1- methylethyl)-6-(1H-pyrazol-1- yl)pyrido[2,3-d]pyrimidin-7(8H)- one |
| 54 | 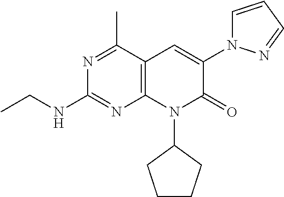 [SMILES: CCNc1nc(C)c2cc(-n3cccn3)c(=O)n(C3CCCC3)c2n1] | 8-cyclopentyl-2-(ethylamino)-4- methyl-6-(1H-pyrazol-1- yl)pyrido[2,3-d]pyrimidin-7(8H)- one |
| 55 | 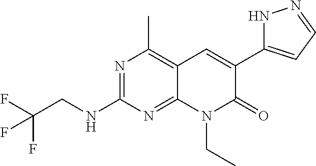 [SMILES: CCn1c(=O)c(-c2ccn[nH]2)cc2c(C)nc(NCC(F)(F)F)nc21] | 8-ethyl-4-methyl-6-(1H-pyrazol-5- yl)-2-[(2,2,2- trifluoroethyl)amino]pyrido[2,3- d]pyrimidin-7(8H)-one |
| 56 | 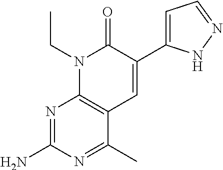 [SMILES: CCn1c(=O)c(-c2ccn[nH]2)cc2c(C)nc(N)nc21] | 2-amino-8-ethyl-4-methyl-6-(1H- pyrazol-5-yl)pyrido[2,3- d]pyrimidin-7(8H)-one |
| 57 | 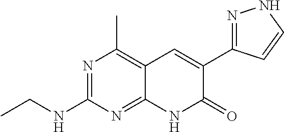 [SMILES: CCNc1nc(C)c2cc(-c3cc[nH]n3)c(=O)[nH]c2n1] | 2-(ethylamino)-4-methyl-6-(1H- pyrazol-3-yl)pyrido[2,3- d]pyrimidin-7(8H)-one |
| 58 | 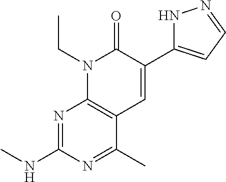 [SMILES: CCn1c(=O)c(-c2ccn[nH]2)cc2c(C)nc(NC)nc21] | 8-ethyl-4-methyl-2-(methylamino)- 6-(1H-pyrazol-5-yl)pyrido[2,3- d]pyrimidin-7(8H)-one |
| 59 | 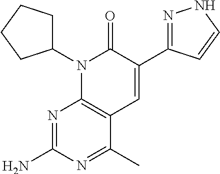 [SMILES: Cc1nc(N)nc2c1cc(-c1cc[nH]n1)c(=O)n2C1CCCC1] | 2-amino-8-cyclopentyl-4-methyl-6- (1H-pyrazol-3-yl)pyrido[2,3- d]pyrimidin-7(8H)-one |
| 60 | 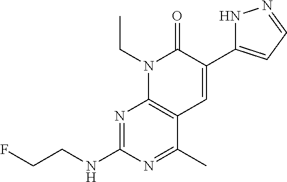 [SMILES: CCn1c(=O)c(-c2ccn[nH]2)cc2c(C)nc(NCCF)nc21] | 8-ethyl-2-[(2-fluoroethyl)amino]-4- methyl-6-(1H-pyrazol-5- yl)pyrido[2,3-d]pyrimidin-7(8H)- one |
| 61 | 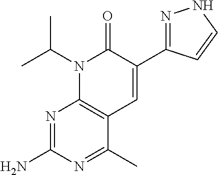 [SMILES: Cc1nc(N)nc2c1cc(-c1cc[nH]n1)c(=O)n2C(C)C] | 2-amino-4-methyl-8-(1- methylethyl)-6-(1H-pyrazol-3- yl)pyrido[2,3-d]pyrimidin-7(8H)- one |
| 62 | 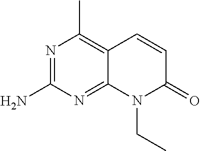 | 2-amino-8-ethyl-4- methylpyrido[2,3-d]pyrimidin- 7(8H)-one |
| 63 | 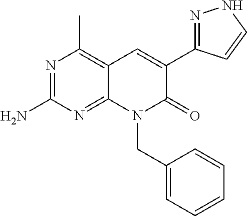 [SMILES: Cc1nc(N)nc2c1cc(-c1cc[nH]n1)c(=O)n2Cc1ccccc1] | 2-amino-4-methyl-8- (phenylmethyl)-6-(1H-pyrazol-3- yl)pyrido[2,3-d]pyrimidin-7(8H)- one |
| 64 | 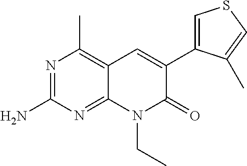 [SMILES: CCn1c(=O)c(-c2cscc2C)cc2c(C)nc(N)nc21] | 2-amino-8-ethyl-4-methyl-6-(4- methyl-3-thienyl)pyrido[2,3- d]pyrimidin-7(8H)-one |
| 65 | 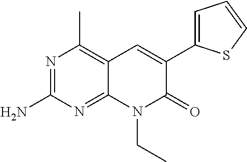 [SMILES: CCn1c(=O)c(-c2cccs2)cc2c(C)nc(N)nc21] | 2-amino-8-ethyl-4-methyl-6-(2- thienyl)pyrido[2,3-d]pyrimidin- 7(8H)-one |
| 66 | 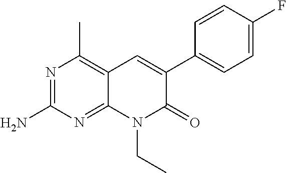 [SMILES: CCn1c(=O)c(-c2ccc(F)cc2)cc2c(C)nc(N)nc21] | 2-amino-8-ethyl-6-(4- fluorophenyl)-4-methylpyrido[2,3- d]pyrimidin-7(8H)-one |
| 67 | 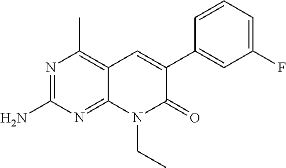 [SMILES: CCn1c(=O)c(-c2cccc(F)c2)cc2c(C)nc(N)nc21] | 2-amino-8-ethyl-6-(3- fluorophenyl)-4-methylpyrido[2,3- d]pyrimidin-7(8H)-one |
| 68 | 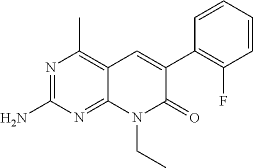 [SMILES: CCn1c(=O)c(-c2ccccc2F)cc2c(C)nc(N)nc21] | 2-amino-8-ethyl-6-(2- fluorophenyl)-4-methylpyrido[2,3- d]pyrimidin-7(8H)-one |
| 69 | 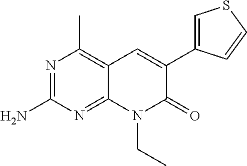 [SMILES: CCn1c(=O)c(-c2ccsc2)cc2c(C)nc(N)nc21] | 2-amino-8-ethyl-4-methyl-6-(3- thienyl)pyrido[2,3-d]pyrimidin- (8H)-one |
| 70 | 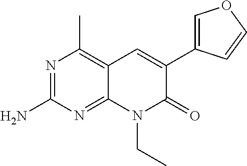 [SMILES: CCn1c(=O)c(-c2ccoc2)cc2c(C)nc(N)nc21] | 2-amino-8-ethyl-6-furan-3-yl-4- methylpyrido[2,3-d]pyrimidin- 7(8H)-one |
| 71 | 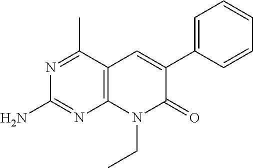 [SMILES: CCn1c(=O)c(-c2ccccc2)cc2c(C)nc(N)nc21] | 2-amino-8-ethyl-4-methyl-6- phenylpyrido[2,3-d]pyrimidin- 7(8H)-one |
| 72 | 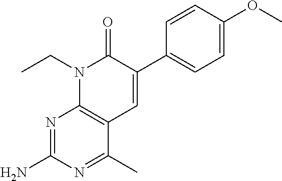 [SMILES: CCn1c(=O)c(-c2ccc(OC)cc2)cc2c(C)nc(N)nc21] | 2-amino-8-ethyl-4-methyl-6-[4- (methyloxy)phenyl]pyrido[2,3- d]pyrimidin-7(8H)-one |
| 73 | 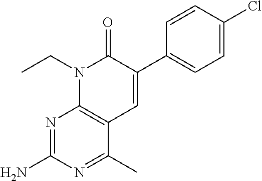 [SMILES: CCn1c(=O)c(-c2ccc(Cl)cc2)cc2c(C)nc(N)nc21] | 2-amino-6-(4-chlorophenyl)-8- ethyl-4-methylpyrido[2,3- d]pyrimidin-7(8H)-one |
| 74 | 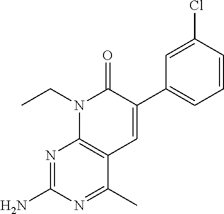 [SMILES: CCn1c(=O)c(-c2cccc(Cl)c2)cc2c(C)nc(N)nc21] | 2-amino-6-(3-chlorophenyl)-8- ethyl-4-methylpyrido[2,3- d]pyrimidin-7(8H)-one |
| 75 | 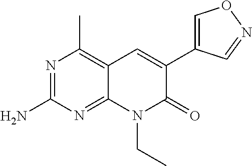 [SMILES: CCn1c(=O)c(-c2cnoc2)cc2c(C)nc(N)nc21] | 2-amino-8-ethyl-6-isoxazol-4-yl-4- methylpyrido[2,3-d]pyrimidin- 7(8H)-one |
| 76 | 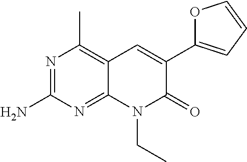 [SMILES: CCn1c(=O)c(-c2ccco2)cc2c(C)nc(N)nc21] | 2-amino-8-ethyl-6-furan-2-yl-4- methylpyrido[2,3-d]pyrimidin- 7(8H)-one |
| 77 | 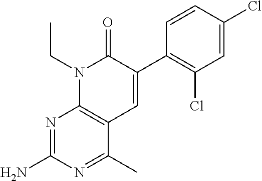 [SMILES: CCn1c(=O)c(-c2ccc(Cl)cc2Cl)cc2c(C)nc(N)nc21] | 2-amino-6-(2,4-dichlorophenyl)-8- ethyl-4-methylpyrido[2,3- d]pyrimidin-7(8H)-one |
| 78 | 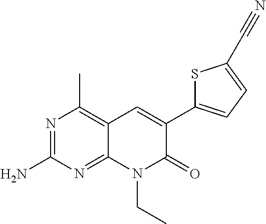 [SMILES: CCn1c(=O)c(-c2ccc(C#N)s2)cc2c(C)nc(N)nc21] | (2-amino-8-ethyl-4-methyl-7-oxo- 7,8-dihydropyrido[2,3-d]pyrimidin- 6-yl)thiophene-2-carbonitrile |
| 79 | 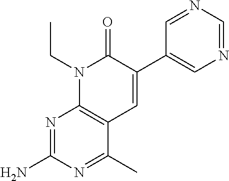 [SMILES: CCn1c(=O)c(-c2cncnc2)cc2c(C)nc(N)nc21] | 2-amino-8-ethyl-4-methyl-6- pyrimidin-5-ylpyrido[2,3- d]pyrimidin-7(8H)-one |
| 80 | 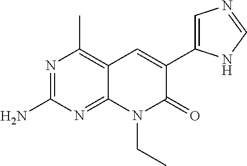 [SMILES: CCn1c(=O)c(-c2cnc[nH]2)cc2c(C)nc(N)nc21] | 2-amino-8-ethyl-6-(1H-imidazol-5- yl)-4-methylpyrido[2,3- d]pyrimidin-7(8H)-one |
| 81 | 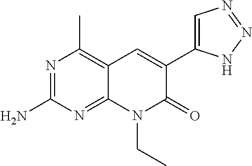 [SMILES: CCn1c(=O)c(-c2cnn[nH]2)cc2c(C)nc(N)nc21] | 2-amino-8-ethyl-4-methyl-6-(1H- 1,2,3-triazol-5-yl)pyrido[2,3- d]pyrimidin-7(8H)-one |
| 82 | 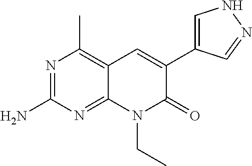 [SMILES: CCn1c(=O)c(-c2cn[nH]c2)cc2c(C)nc(N)nc21] | 2-amino-8-ethyl-4-methyl-6-(1H- pyrazol-4-yl)pyrido[2,3- d]pyrimidin-7(8H)-one |
| 83 | 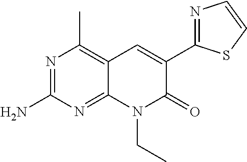 [SMILES: CCn1c(=O)c(-c2nccs2)cc2c(C)nc(N)nc21] | 2-amino-8-ethyl-4-methyl-6-(1,3- thiazol-2-yl)pyrido[2,3- d]pyrimidin-7(8H)-one |
| 84 | 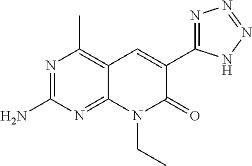 [SMILES: CCn1c(=O)c(-c2nnn[nH]2)cc2c(C)nc(N)nc21] | 2-amino-8-ethyl-4-methyl-6-(1H- tetrazol-5-yl)pyrido[2,3- d]pyrimidin-7(8H)-one |
| 85 | 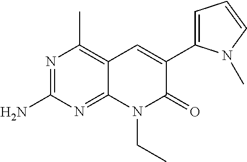 [SMILES: CCn1c(=O)c(-c2cccn2C)cc2c(C)nc(N)nc21] | 2-amino-8-ethyl-4-methyl-6-(1- methyl-1H-pyrrol-2-yl)pyrido[2,3- d]pyrimidin-7(8H)-one |
| 86 | 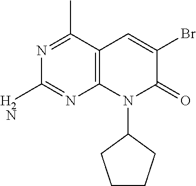 [SMILES: Cc1nc(N)nc2c1cc(Br)c(=O)n2C1CCCC1] | 2-amino-6-bromo-8-cyclopentyl-4- methylpyrido[2,3-d]pyrimidin- 7(8H)-one |
| 87 | 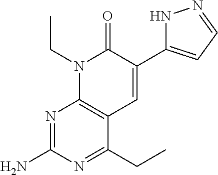 [SMILES: CCc1nc(N)nc2c1cc(-c1ccn[nH]1)c(=O)n2CC] | 2-amino-4,8-diethyl-6-(1H-pyrazol- 5-yl)pyrido[2,3-d]pyrimidin-7(8H)- one |
| 88 | 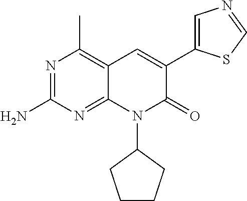 [SMILES: Cc1nc(N)nc2c1cc(-c1cncs1)c(=O)n2C1CCCC1] | 2-amino-8-cyclopentyl-4-methyl-6- (1,3-thiazol-5-yl)pyrido[2,3- d]pyrimidin-7(8H)-one |

### Compounds of Formula IA

In another embodiment, the compound of Formula I is a compound of Formula

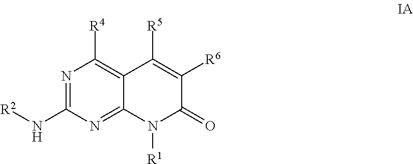

or a pharmaceutically acceptable salt thereof, wherein:

R¹ is alkyl, cycloalkyl, cycloalkylalkyl, aryl, arylalkyl, heterocycloalkyl, heterocycloalkylalkyl, heteroaryl or heteroarylalkyl;

R² is hydrogen or alkyl;

R⁴ is alkyl;

R⁵ is hydrogen;

R⁶ is phenyl, acyl, or heteroaryl wherein the phenyl and heteroaryl are is optionally substituted with 1, 2, 3, 4, or 5 R⁹ groups; and each R⁹, when present, is independently halo, alkyl, haloalkyl, alkoxy, haloalkoxy, cyano, amino, alkylamino, dialkylamino, alkoxyalkyl, carboxyalkyl, alkoxycarbonyl, aminoalkyl, cycloalkyl, aryl, arylalkyl, aryloxy, heterocycloalkyl, or heteroaryl and where the cycloalkyl, aryl, heterocycloalkyl, and heteroaryl, each either alone or as part of another group within R⁹, are independently optionally substituted with 1, 2, 3, or 4 groups selected from halo, alkyl, haloalkyl, hydroxy, alkoxy, haloalkoxy, amino, alkylamino, and dialkylamino.

In one embodiment, where R¹ is alkyl, cycloalkyl, heterocycloalkylalkyl, or arylalkyl; X is —NH—; R² is hydrogen or alkyl; R⁴ is alkyl; R⁵ is hydrogen; R⁶ is phenyl or heteroaryl wherein the phenyl and heteroaryl are is optionally substituted with one, two, or three R⁹ groups; each R⁸, when present, is independently amino, alkylamino, dialkylamino, or halo; and each R⁸, when present, is independently alkyl, arylalkyl, cyano, aryl, alkoxycarbonyl.

In another embodiment, R⁴ is methyl.

In another embodiment. R¹ is alkyl, cycloalkyl, or heterocycloalkyl.

In another embodiment, R¹ is alkyl.

In another embodiment, R⁶ is heteroaryl optionally substituted with 1, 2, or 3 R⁹ groups.

In another embodiment, each R⁹, when present, is independently alkyl, arylalkyl, cyano, aryl, alkoxycarbonyl, or halo.

In another embodiment, le is pyrazolyl, imidazolyl, thienyl, thiazolyl, oxazolyl, isoxazolyl, oxadiazolyl, furanyl, pyrrolyl, triazolyl, or tetrazolyl; each of which is optionally substituted with 1, 2, or 3 R⁹ groups.

In another embodiment, R⁶ is pyrazol-3-yl, pyrazol-4-yl, pyrazol-5-yl, imidazol-2-yl, imidazol-4-yl, imidazol-5-yl, thien-2-yl, thien-3-yl, thiazol-2-yl, thiazol-4-yl, thiazol-5-yl, oxazol-2-yl, oxazol-5-yl, isoxazol-3-yl, isoxazol-4-yl, isoxazol-5-yl, 1,2,3-oxadiazol-4-yl, 1,2,3-oxadiazol-5-yl, 1,3,4-oxadiazol-2-yl, 1,2,4-oxadiazol-3-yl, 1,2,4-oxadiazol-5-yl, furan-2-yl, furan-3-yl, pyrrol-2-yl, pyrrol-3-yl, triazol-4-yl, triazol-5 yl, or tetrazol-5-yl; each of which is optionally substituted with 1, 2, or 3 R⁹ groups.

In another embodiment, R⁶ is pyrazinyl, pyrimidinyl, or pyridazinyl each of which is optionally substituted with 1, 2, or 3 R⁹ groups and R⁴ is methyl.

In another embodiment, R² is hydrogen, R⁴ is methyl, R¹ is optionally substituted alkyl, cycloalkyl, or heterocycloalkyl, and R⁶ is heteroaryl optionally substituted with 1, 2, or 3 R⁹ groups.

In another embodiment, the compound of Formula IA is selected from:

8-ethyl-2-(ethylamino)-4-methyl-6-phenylpyrido
[2,3-d]pyrimidin-7(8H)-one;
6-biphenyl-4-yl-8-ethyl-2-(ethylamino)-4-
methylpyrido[2,3-d]pyrimidin-7(8H)-one;
6-(2,4-difluorophenyl)-8-ethyl-2-(ethylamino)-4-
methylpyrido[2,3-d]pyrimidin-7(8H)-one;
6-(3-chloro-4-fluorophenyl)-8-ethyl-2-(ethylamino)-4-
methylpyrido[2,3-d]pyrimidin-7(8H)-one;
8-ethyl-2-(ethylamino)-4-methyl-6-[4-
(methyloxy)phenyl]pyrido[2,3-d]pyrimidin-7(8H)-one;
6-(2,4-dichlorophenyl)-8-ethyl-2-(ethylamino)-4-
methylpyrido[2,3-d]pyrimidin-7(8H)-one;
6-(3,4-difluorophenyl)-8-ethyl-2-(ethylamino)-4-
methylpyrido[2,3-d]pyrimidin-7(8H)-one;
8-ethyl-2-(ethylamino)-4-methyl-6-[2-
(methyloxy)phenyl]pyrido[2,3-d]pyrimidin-7(8H)-one;
8-ethyl-2-(ethylamino)-4-methyl-6[4-
(phenyloxy)phenyl]pyrido[2,3-d]pyrimidin-7(8H)-one;
6-[2,4-bis(methyloxy)phenyl]-8-ethyl-2-(ethylamino)-
4-methylpyrido[2,3-d]pyrimidin-7(8H)-one;
8-ethyl-2-(ethylamino)-6-(3-fluorophenyl)-4-
methylpyrido[2,3-d]pyrimidin-7(8H)-one;
8-ethyl-2-(ethylamino)-6-(2-fluorophenyl)-4-
methylpyrido[2,3-d]pyrimidin-7(8H)-one;
8-ethyl-2-(ethylamino)-4-methyl-6-[3-
(trifluoromethyl)phenyl]pyrido[2,3-d]pyrimidin-7(8H)-one;
8-ethyl-2-(ethylamino)-6-(4-fluorophenyl)-4-
methylpyrido[2,3-d]pyrimidin-7(8H)-one;
8-ethyl-2-(ethylamino)-4-methyl-6-
(2-thienyl)pyrido[2,3-d]pyrimidin-7(8H)-one;
8-ethyl-2-(ethylamino)-4-methyl-6-[3-
(methyloxy)phenyl]pyrido[2,3-d]pyrimidin-7(8H)-one;
6-(3-chlorophenyl)-8-ethyl-2-(ethylamino)-4-
methylpyrido[2,3-d]pyrimidin-7(8H)-one;
6-(4-chlorophenyl)-8-ethyl-2-(ethylamino)-4-
methylpyrido[2,3-d]pyrimidin-7(8H)-one;
8-ethyl-2-(ethylamino)-4-methyl-6-
(3-thienyl)pyrido[2,3-d]pyrimidin-7(8H)-one;
8-ethyl-2-(ethylamino)-4-methyl-6-(4-methyl-2-
thienyl)pyrido[2,3-d]pyrimidin-7(8H)-one;
8-ethyl-2-(ethylamino)-4-methyl-6-(4-methyl-3-
thienyl)pyrido[2,3-d]pyrimidin-7(8H)-one;
1,1-dimethylethyl 2-[8-ethyl-2-(ethylamino)-4-methyl-7-
oxo-7,8-dihydropyrido[2,3-d]pyrimidin-6-yl]-1H-pyrrole-
1-carboxylate:
8-ethyl-2-(ethylamino)-4-methyl-6-(1H-
pyrrol-2-yl)pyrido[2,3-d]pyrimidin-7(8H)-one;
6-(5-chloro-2-thienyl)-8-ethyl-2-
(ethylamino)-4-methylpyrido[2,3-d]pyrimidin-7(8H)-one;
8-ethyl-2-(ethylamino)-4-methyl-6-
pyrimidin-5-ylpyrido[2,3-d]pyrimidin-7(8H)-one;
8-ethyl-2-(ethylamino)-6-(3-fluoropyridn-4-yl)-4-
methylpyrido[2,3-d]pyrimidin-7(8H)-one;
8-ethyl-2-(ethylamino)-6-furan-3-yl-4-
methylpyrido[2,3-d]pyrimidin-7(8H)-one;
8-ethyl-2-(ethylamino)-4-methyl-6-[1-(phenylmethyl)-
1H-pyrazol-4-yl]pyrido[2,3-d]pyrimidin-7(8H)-one;
2-(ethylamino)-4-methyl-8-(1-methylethyl)-6-
(2-thienyl)pyrido[2,3-d]pyrimidin-7(8H)-one;
8-ethyl-2-(ethylamino)-6-(1H-indol-6-yl)-4-
methylpyrido[2,3-d]pyrimidin-7(8H)-one;
8-ethyl-2-(ethylamino)-4-methyl-6-(5-phenyl-2-
thienyl)pyrido[2,3-d]pyrimidin-7(8H)-one;
2-(ethylamino)-6-furan-3-yl-4-methyl-8-(1-
methylethyl)pyrido[2,3-d]pyrimidin-7(8H)-one;
8-ethyl-2-(ethylamino)-4-methyl-6-(1H-pyrazol-5-
yl)pyrido[2,3-d]pyrimidin-7(8H)-one;
8-cyclohexyl-2-(ethylamino)-4-methyl-6-(2-
thienyl)pyrido[2,3-d]pyrimidin-7(8H)-one;
8-ethyl-2-(ethylamino)-4-methyl-6-(1,3-
thiazol-2-yl)pyrido[2,3-d]pyrimidin-7(8H)-one;
8-cyclopentyl-2-(ethylamino)-4-methyl-6-(1H-
pyrazol-3-yl)pyrido[2,3-d]pyrimidin-7(8H)-one;
2-(ethylamino)-4-methyl-8-(1-methylethyl)-6-(1H-
pyrazol-5-yl)pyrido[2,3-d]pyrimidin-7(8H)-one;
8-ethyl-2-(ethylamino)-4-methyl-6-(1H-
pyrazol-1-yl)pyrido[2,3-d]pyrimidin-7(8H)-one;
2-(ethylamino)-4-methyl-8-(1-methylethyl)-6-(1H-
pyrazol-1-yl)pyrido[2,3-d]pyrimidin-7(8H)-one;
8-cyclopentyl-2-(ethylamino)-4-methyl-6-(1H-
pyrazol-1-yl)pyrido[2,3-d]pyrimidin-7(8H)-one;
8-ethyl-4-methyl-6-(1H-pyrazol-5-yl)-2-[(2,2,2-
trifluoroethyl)amino]pyrido[2,3-d]pyrimidin-7(8H)-one;
2-amino-8-ethyl-4-methyl-6-(1H-
pyrazol-5-yl)pyrido[2,3-d]pyrimidin-7(8H)-one;
2-(ethylamino)-4-methyl-6-(1H-
pyrazol-3-yl)pyrido[2,3-d]pyrimidin-7(8H)-one;
8-ethyl-4-methyl-2-(methylamino)-6-(1H-
pyrazol-5-yl)pyrido[2,3-d]pyrimidin-7(8H)-one;
2-amino-8-cyclopentyl-4-methyl-6-(1H-
pyrazol-3-yl)pyrido[2,3-d]pyrimidin-7(8H)-one;
8-ethyl-2-[(2-fluoroethyl)amino]-4-methyl-6-(1H-
pyrazol-5-yl)pyrido[2,3-d]pyrimidin-7(8H)-one;
2-amino-4-methyl-8-(1-methylethyl)-6-(1H-
pyrazol-3-yl)pyrido[2,3-d]pyrimidin-7(8H)-one;
2-amino-4-methyl-8-(phenylmethyl)-6-(1H-
pyrazol-3-yl)pyrido[2,3-d]pyrimidin-7(8H)-one;
2-amino-8-ethyl-4-methyl-6-(4-methyl-3-
thienyl)pyrido[2,3-d]pyrimidin-7(8H)-one;
2-amino-8-ethyl-4-methyl-6-(2-
thienyl)pyrido[2,3-d]pyrimidin-7(8H)-one;
2-amino-8-ethyl-6-(4-fluorophenyl)-4-
methylpyrido[2,3-d]pyrimidin-7(8H)-one;
2-amino-8-ethyl-6-(3-fluorophenyl)-4-
methylpyrido[2,3-d]pyrimidin-7(8H)-one;
2-amino-8-ethyl-6-(2-fluorophenyl)-4-
methylpyrido[2,3-d]pyrimidin-7(8H)-one;
2-amino-8-ethyl-4-methyl-6-(3-
thienyl)pyrido[2,3-d]pyrimidin-7(8H)-one;
2-amino-8-ethyl-6-furan-3-yl-4-
methylpyrido[2,3-d]pyrimidin-7(8H)-one;
2-amino-8-ethyl-4-methyl-6-
phenylpyrido[2,3-d]pyrimidin-7(8H)-one;
2-amino-8-ethyl-4-methyl-6-[4-
(methyloxy)phenyl]pyrido[2,3-d]pyrimidin-7(8H)-one;
2-amino-6-(4-chlorophenyl)-8-ethyl-4-
methylpyrido[2,3-d]pyrimidin-7(8H)-one;
2-amino-6-(3-chlorophenyl)-8-ethyl-4-
methylpyrido[2,3-d]pyrimidin-7(8H)-one;
2-amino-8-ethyl-6-isoxazol-4-yl-4-
methylpyrido[2,3-d]pyrimidin-7(8H)-one;
2-amino-8-ethyl-6-furan-2-yl-4-
methylpyrido[2,3-d]pyrimidin-7(8H)-one;
2-amino-6-(2,4-dichlorophenyl)-8-ethyl-4-
methylpyrido[2,3-d]pyrimidin-7(8H)-one;
5-(2-amino-8-ethyl-4-methyl-7-oxo-7,8-dihydropyrido[2,3-
d]pyrimidin-6-yl)thiophene-2-carbonitrile:
2-amino-8-ethyl-4-methyl-6-pyrimidin-5-
ylpyrido[2,3-d]pyrimidin-7(8H)-one;
2-amino-8-ethyl-6-(1H-imidazol-5-yl)-4-
methylpyrido[2,3-d]pyrimidin-7(8H)-one;
2-amino-8-ethyl-4-methyl-6-(1H-1,2,3-
triazol-5-yl)pyrido[2,3-d]pyrimidin-7(8H)-one;
2-amino-8-ethyl-4-methyl-6-(1H-pyrazol-4-
yl)pyrido[2,3-d]pyrimidin-7(8H)-one;
2-amino-8-ethyl-4-methyl-6-(1,3-
thiazol-2-yl)pyrido[2,3-d]pyrimidin-7(8H)-one;
2-amino-8-ethyl-4-methyl-6-(1H-tetrazol-
5-yl)pyrido[2,3-d]pyrimidin-7(8H)-one;
2-amino-8-ethyl-4-methyl-6-(1-methyl-1H-
pyrrol-2-yl)pyrido[2,3-d]pyrimidin-7(8H)-one;
2-amino-4,8-diethyl-6-(1H-pyrazol-5-
yl)pyrido[2,3-d]pyrimidin-7(8H)-one; and
2-amino-8-cyclopentyl-4-methyl-6-(1,3-
thiazol-5-yl)pyrido[2,3-d]pyrimidin-7(8H)-one.

In another embodiment, the compound of Formula IA is selected from:

8-ethyl-2-(ethylamino)-4-methyl-6-
(2-thienyl)pyrido[2,3-d]pyrimidin-7(8H)-one;
8-ethyl-2-(ethylamino)-4-methyl-6-(3-
thienyl)pyrido[2,3-d]pyrimidin-7(8H)-one;
8-ethyl-2-(ethylamino)-4-methyl-6-(4-methyl-2-
thienyl)pyrido[2,3-d]pyrimidin-7(8H)-one;
8-ethyl-2-(ethylamino)-4-methyl-6-(4-methyl-3-
thienyl)pyrido[2,3-d]pyrimidin-7(8H)-one;
1,1-dimethylethyl 2-[8-ethyl-2-(ethylamino)-4-methyl-7-oxo-7,8-
dihydropyrido[2,3-d]pyrimidin-6-yl]-1H-pyrrole-1-carboxylate
8-ethyl-2-(ethylamino)-4-methyl-6-(1H-pyrrol-2-yl)pyrido[2,3-
d]pyrimidin-7(8H)-one;
6-(5-chloro-2-thienyl)-8-ethyl-2-(ethylamino)-4-
methylpyrido[2,3-d]pyrimidin-7(8H)-one;
8-ethyl-2-(ethylamino)-4-methyl-6-pyrimidin-5-
ylpyrido[2,3-d]pyrimidin-7(8H)-one;
8-ethyl-2-(ethylamino)-6-(3-fluoropyridn-4-yl)-4-
methylpyrido[2,3-d]pyrimidin-7(8H)-one;
8-ethyl-2-(ethylamino)-6-furan-3-yl-4-
methylpyrido[2,3-d]pyrimidin-7(8H)-one;
2-(ethylamino)-4-methyl-8-(1-methylethyl)-6-(2-
thienyl)pyrido[2,3-d]pyrimidin-7(8H)-one;
8-ethyl-2-(ethylamino)-6-(1H-indol-6-yl)-4-
methylpyrido[2,3-d]pyrimidin-7(8H)-one;
8-ethyl-2-(ethylamino)-4-methyl-6-(5-phenyl-2-
thienyl)pyrido[2,3-d]pyrimidin-7(8H)-one;
2-(ethylamino)-6-furan-3-yl-4-methyl-8-(1-
methylethyl)pyrido[2,3-d]pyrimidin-7(8H)one;
8-ethyl-2-(ethylamino)-4-methyl-6-(1H-
pyrazol-5-yl)pyrido[2,3-d]pyrimidin-7(8H)-one;
8-cyclohexyl-2-(ethylamino)-4-methyl-6-(2-
thienyl)pyrido[2,3-d]pyrimidin-7(8H)-one;
8-ethyl-2-(ethylamino)-4-methyl-6-(1,3-thiazol-2-
yl)pyrido[2,3-d]pyrimidin-7(8H)-one;
8-cyclopentyl-2-(ethylamino)-4-methyl-6-(1H-pyrazol-
3-yl)pyrido[2,3-d]pyrimidin-7(8H)-one;
2-(ethylamino)-4-methyl-8-(1-methylethyl)-6-(1H-pyrazol-5-
yl)pyrido[2,3-d]pyrimidin-7(8H)-one;
8-ethyl-2-(ethylamino)-4-methyl-6-(1H-pyrazol-1-
yl)pyrido[2,3-d]pyrimidin-7(8H)-one;
2-(ethylamino)-4-methyl-8-(1-methylethyl)-6-(1H-pyrazol-1-
yl)pyrido[2,3-d]pyrimidin-7(8H)-one;
8-cyclopentyl-2-(ethylamino)-4-methyl-6-(1H-pyrazol-1-
yl)pyrido[2,3-d]pyrimidin-7(8H)-one;
8-ethyl-4-methyl-6-(1H-pyrazol-5-yl)-2-[(2,2,2-
trifluoroethyl)amino]pyrido[2,3-d]pyrimidin-7(8H)-one;
2-(ethylamino)-4-methyl-6-(1H-pyrazol-3-
yl)pyrido[2,3-d]pyrimidin-7(8H)-one;
8-ethyl-4-methyl-2-(methylamino)-6-(1H-pyrazol-5-
yl)pyrido[2,3-d]pyrimidin-7(8H)-one;and
8-ethyl-2-[(2-fluoroethyl)amino]-4-methyl-6-(1H-pyrazol-5-
yl)pyrido[2,3-d]pyrimidin-7(8H)-one.

in another embodiment, the compound of Formula IA is selected form:

2-amino-8-ethyl-4-methyl-6-(1H-pyrazol-5-
yl)pyrido [2,3-d]pyrimidin-7(8H)-one;
2-amino-8-cyclopentyl-4-methyl-6-(1H-pyrazol-3-
yl)pyrido[2,3-d]pyrimidin-7(8H)-one;
2-amino-4-methyl-8-(1-methylethyl)-6-(1H-pyrazol-3-
yl)pyrido[2,3-d]pyrimidin-7(8H)-one;
2-amino-4-methyl-8-(phenylmethyl)-6-(1H-pyrazol-3-
yl)pyrido[2,3-d]pyrimidin-7(8H)-one;
2-amino-8-ethyl-4-methyl-6-(4-methyl-3-
thienyl)pyrido[2,3-d]pyrimidin-7(8H)-one;
2-amino-8-ethyl-4-methyl-6-(2-
thienyl)pyrido[2,3-d]pyrimidin-7(8H)-one;
2-amino-8-ethyl-4-methyl-6-(3-
thienyl)pyrido[2,3-d]pyrimidin-7(8H)-one;
2-amino-8-ethyl-6-furan-3-yl-4-
methylpyrido[2,3-d]pyrimidin-7(8H)-one;
2-amino-8-ethyl-4-methyl-6-
phenylpyrido[2,3-d]pyrimidin-7(8H)-one;
2-amino-8-ethyl-6-isoxazol-4-yl-4-
methylpyrido[2,3-d]pyrimidin-7(8H)-one;
2-amino-8-ethyl-6-furan-2-yl-4-
methylpyrido[2,3-d]pyrimidin-7(8H)-one;
5-(2-amino-8-ethyl-4-methyl-7-oxo-7,8-
dihydropyrido[2,3-d]pyrimidin-6-yl)thiophene-2-carbonitrile;
2-amino-8-ethyl-4-methyl-6-pyrimidin-5-
ylpyrido[2,3-d]pyrimidin-7(8H)-one;
2-amino-8-ethyl-6-(1H-imidazol-5-yl)-4-
methylpyrido[2,3-d]pyrimidin-7(8H)-one;
2-amino-8-ethyl-4-methyl-6-(1H-1,2,3-triazol-5-
yl)pyrido[2,3-d]pyrimidin-7(8H)-one;
2-amino-8-ethyl-4-methyl-6-(1H-pyrazol-4-
yl)pyrido[2,3-d]pyrimidin-7(8H)-one;
2-amino-8-ethyl-4-methyl-6-(1,3-thiazol-2-
yl)pyrido[2,3-d]pyrimidin-7(8H)-one;
2-amino-8-ethyl-4-methyl-6-(1H-tetrazol-5-
yl)pyrido[2,3-d]pyrimidin-7(8H)-one;
2-amino-8-ethyl-4-methyl-6-(1-methyl-1H-
pyrrol-2-yl)pyrido[2,3-d]pyrimidin-7(8H)-one;
2-amino-4,8-diethyl-6-(1H-pyrazol-5-
yl)pyrido[2,3-d]pyrimidin-7(8H)-one; and
2-amino-8-cyclopentyl-4-methyl-6-(1,3-thiazol-
5-yl)pyrido[2,3-d]pyrimidin-7(8H)-one.

In another embodiment, the compound of Formula IA is 2-amino-8-ethyl-4-methyl-6-(1H-pyrazol-5-yl)pyrido[2,3-d]pyrimidin-7(8H)-one (Compound A) or a pharmaceutically acceptable salt thereof.

### General Administration

In one aspect, the invention provides pharmaceutical compositions comprising an inhibitor of PI3K and mTOR of Formula I, optionally in combination with one or both of bendamustine and rituximab as described herein, and a pharmaceutically acceptable carrier, excipient or diluent. In certain other specific embodiments, administration is by the oral route. Administration of the compounds of Formula I, or their pharmaceutically acceptable salts, in pure form or in an appropriate pharmaceutical composition in combination with one or both of bendamustine and rituximab as described herein, can be carried out via any of the accepted modes of administration or agents for serving similar utilities. Thus, the Compound of Formula I and with one or both of bendamustine and rituximab can be administered in the same or separate vehicles. Administration can be, for example, orally, nasally, parenterally (intravenous, intramuscular, or subcutaneous), topically, transdermally, intravaginally, intravesically, intracistemally, or rectally, in the form of solid, semi-solid, lyophilized powder, or liquid dosage forms, such as for example, tablets, suppositories, pills, soft elastic and hard gelatin capsules, powders, solutions, suspensions, or aerosols, or the like, specifically in unit dosage forms suitable for simple administration of precise dosages.

The compositions will include a conventional pharmaceutical carrier or excipient and a Compound of Formula I as the/an active agent, optionally with one or both of bendamustine and rituximab, and, in addition, may include carriers and adjuvants, and so on.

Adjuvants include preserving, wetting, suspending, sweetening, flavoring, perfuming, emulsifying, and dispensing agents. Prevention of the action of microorganisms can be ensured by various antibacterial and antifungal agents, for example, parabens, chlorobutanol, phenol, sorbic acid, and the like. It may also be desirable to include isotonic agents, for example sugars, sodium chloride, and the like. Prolonged absorption of the injectable pharmaceutical form can be brought about by the use of agents delaying absorption, for example, aluminum monostearate and gelatin.

If desired, a pharmaceutical composition of the invention may also contain minor amounts of auxiliary substances such as wetting or emulsifying agents, pH buffering agents, antioxidants, and the like, such as, for example, citric acid, sorbitan monolaurate, triethanolamine oleate, butylated hydroxytoluene, etc.

The choice of formulation depends on various factors such as the mode of drug administration (e.g., for oral administration, formulations in the form of tablets, pills or capsules) and the bioavailability of the drug substance. Recently, pharmaceutical formulations have been developed especially for drugs that show poor bioavailability based upon the principle that bioavailability can be increased by increasing the surface area i.e., decreasing particle size. For example, U.S. Pat. No. 4,107,288 describes a pharmaceutical formulation having particles in the size range from 10 to 1,000 nm in which the active material is supported on a crosslinked matrix of macromolecules. U.S. Pat. No. 5,145,684 describes the production of a pharmaceutical formulation in which the drug substance is pulverized to nanoparticles (average particle size of 400 nm) in the presence of a surface modifier and then dispersed in a liquid medium to give a pharmaceutical formulation that exhibits remarkably high bioavailability.

Compositions suitable for parenteral injection may comprise physiologically acceptable sterile aqueous or nonaqueous solutions, dispersions, suspensions or emulsions, and sterile powders for reconstitution into sterile injectable solutions or dispersions. Examples of suitable aqueous and nonaqueous carriers, diluents, solvents or vehicles include water, ethanol, polyols (propyleneglycol, polyethyleneglycol, glycerol, and the like), suitable mixtures thereof, vegetable oils (such as olive oil) and injectable organic esters such as ethyl oleate. Proper fluidity can be maintained, for example, by the use of a coating such as lecithin, by the maintenance of the required particle size in the case of dispersions and by the use of surfactants.

One specific route of administration is oral, using a convenient daily dosage regimen that can be adjusted according to the degree of severity of the disease-state to be treated.

Solid dosage forms for oral administration include capsules, tablets, pills, powders, and granules. In such solid dosage forms, the active compound is admixed with at least one inert customary excipient (or carrier) such as sodium citrate or dicalcium phosphate or (a) fillers or extenders, as for example, starches, lactose, sucrose, glucose, mannitol, and silicic acid, (b) binders, as for example, cellulose derivatives, starch, alignates, gelatin, polyvinylpyrrolidone, sucrose, and gum acacia, (c) humectants, as for example, glycerol, (d) disintegrating agents, as for example, agar-agar, calcium carbonate, potato or tapioca starch, alginic acid, croscarmellose sodium, complex silicates, and sodium carbonate, (e) solution retarders, as for example paraffin, (f) absorption accelerators, as for example, quaternary ammonium compounds, (g) wetting agents, as for example, cetyl alcohol, and glycerol monostearate, magnesium stearate and the like (h) adsorbents, as for example, kaolin, and bentonite, and (i) lubricants, as for example, talc, calcium stearate, magnesium stearate, solid polyethylene glycols, sodium lauryl sulfate, or mixtures thereof. In the case of capsules, tablets, and pills, the dosage forms may also comprise buffering agents.

Solid dosage forms as described above can be prepared with coatings and shells, such as enteric coatings and others well known in the art. They may contain pacifying agents, and can also be of such composition that they release the active compound or compounds in a certain part of the intestinal tract in a delayed manner. Examples of embedded compositions that can be used are polymeric substances and waxes. The active compounds can also be in microencapsulated form, if appropriate, with one or more of the above-mentioned excipients.

Liquid dosage forms for oral administration include pharmaceutically acceptable emulsions, solutions, suspensions, syrups, and elixirs. Such dosage forms are prepared, for example, by dissolving, dispersing, etc., a compound(s) of the invention, or a pharmaceutically acceptable salt thereof, and optional pharmaceutical adjuvants in a carrier, such as, for example, water, saline, aqueous dextrose, glycerol, ethanol and the like; solubilizing agents and emulsifiers, as for example, ethyl alcohol, isopropyl alcohol, ethyl carbonate, ethyl acetate, benzyl alcohol, benzyl benzoate, propyleneglycol, 1,3-butyleneglycol, dimethylformamide; oils, in particular, cottonseed oil, groundnut oil, corn germ oil, olive oil, castor oil and sesame oil, glycerol, tetrahydrofurfuryl alcohol, polyethyleneglycols and fatty acid esters of sorbitan; or mixtures of these substances, and the like, to thereby form a solution or suspension.

Suspensions, in addition to the active compounds, may contain suspending agents, as for example, ethoxylated isostearyl alcohols, polyoxyethylene sorbitol and sorbitan esters, microcrystalline cellulose, aluminum metahydroxide, bentonite, agar-agar and tragacanth, or mixtures of these substances, and the like.

Compositions for rectal administrations are, for example, suppositories that can be prepared by mixing the compounds of the present invention with for example suitable non-irritating excipients or carriers such as cocoa butter, polyethyleneglycol or a suppository wax, which are solid at ordinary temperatures but liquid at body temperature and therefore, melt while in a suitable body cavity and release the active component therein.

Dosage forms for topical administration of a compound of this invention include ointments, powders, sprays, and inhalants. The active component is admixed under sterile conditions with a physiologically acceptable carrier and any preservatives, buffers, or propellants as may be required. Ophthalmic formulations, eye ointments, powders, and solutions are also contemplated as being within the scope of this invention.

Compressed gases may be used to disperse a compound of this invention in aerosol form. Inert gases suitable for this purpose are nitrogen, carbon dioxide, etc.

Generally, depending on the intended mode of administration, the pharmaceutically acceptable compositions will contain about 1% to about 99% by weight of a compound(s) of the invention, or a pharmaceutically acceptable salt thereof, and 99% to 1% by weight of a suitable pharmaceutical excipient. In one example, the composition will be between about 5% and about 75% by weight of a compound(s) of the invention, or a pharmaceutically acceptable salt thereof, with the rest being suitable pharmaceutical excipients.

Actual methods of preparing such dosage forms are known, or will be apparent, to those skilled in this art; for example, see Remington's Pharmaceutical Sciences, 18th Ed., (Mack Publishing Company, Easton, Pa., 1990). The composition to be administered will, in any event, contain an effective amount of a compound of the invention, or a pharmaceutically acceptable salt thereof, for treatment of a disease-state in accordance with the teachings of this invention.

In the pharmaceutical compositions disclosed herein, the compounds of Formula or their pharmaceutically acceptable salts or solvates, are administered in an effective amount which will vary depending upon a variety of factors including the activity of the specific compound employed, the metabolic stability and length of action of the compound, the age, body weight, general health, sex, diet, mode and time of administration, rate of excretion, drug combination, the severity of the particular disease-states, and the host undergoing therapy. The compounds of Formula I can be administered to a patient at dosage levels in the range of about 0.1 to about 1,000 mg per day. For a normal human adult having a body weight of about 70 kilograms, a dosage in the range of about 0.01 to about 100 mg per kilogram of body weight per day is an example. The specific dosage used, however, can vary. For example, the dosage can depend on a number of factors including the requirements of the patient, the severity of the condition being treated, and the pharmacological activity of the compound being used. The determination of optimum dosages for a particular patient is well known to one of ordinary skill in the art. If formulated as a fixed dose, such combination products employ the compounds of this invention within the dosage range described above and the other pharmaceutically active agent(s) within approved dosage ranges. Compounds of Formula I may alternatively be used sequentially with known pharmaceutically acceptable agent(s) when a combination formulation is inappropriate.

In some embodiments, the effective amount produces at least one therapeutic effect selected from the group consisting of reduction in size of a tumor, reduction in metastasis, complete remission, partial remission, stable disease, increase in overall response rate, or a pathologic complete response. In some embodiments, the effective amount produces an improved clinical benefit rate (CBR=CR (complete remission)+PR (partial remission)+SD (stable disease)≧6 months) as compared to treatment with bendamustine or rituximab administered without. Compound A. In some embodiments, the improvement of clinical benefit rate is about 20% or higher. In some embodiments, the improvement of clinical benefit rate is at least about 20%, 30%, 40%, 50%, 60%, 70%, 80%, or more. In some embodiments, the therapeutic effect is an increase in overall response rate. In some embodiments, the increase in overall response rate is about 10%, 20%, 30%, 40%, 50%, 60%, 70%, 80% or more.

In some embodiments, a comparable clinical benefit rate (CBR=CR (complete remission)+PR (partial remission)+SD (stable disease)≧6 cycles) is obtained with treatment of a) Compound A or a pharmaceutically acceptable salt thereof, and either (b) bendamustine or a pharmaceutically acceptable salt thereof or (c) rituximab or (d) a combination of bendamustine or a pharmaceutically acceptable salt thereof and rituximab, as compared to treatment with bendamustine or a pharmaceutically acceptable salt thereof or rituximab or a combination of bendamustine or a pharmaceutically acceptable salt thereof and rituximab, administered without Compound A. In some embodiments, the improvement of clinical benefit rate is at least about 20%. In some embodiments, the improvement of clinical benefit rate is at least about 30%. In some embodiments, the improvement of clinical benefit rate is at least about 40%. In some embodiments, the improvement of clinical benefit rate is at least about 50%. In some embodiments, the improvement of clinical benefit rate is at least about 60%. In some embodiments, the improvement of clinical benefit rate is at least about 70%. In some embodiments, the improvement of clinical benefit rate is at least about 80%.

In some embodiments, a comparable clinical benefit rate (CBR=CR (complete remission)+PR (partial remission)+SD (stable disease)≧6 months) is obtained with treatment of a) Compound A or a pharmaceutically acceptable salt thereof, and either (b) bendamustine or a pharmaceutically acceptable salt thereof or (c) rituximab or (d) a combination of bendamustine or a pharmaceutically acceptable salt thereof and rituximab, as compared to treatment with bendamustine or a pharmaceutically acceptable salt thereof or rituximab or a combination of bendamustine or a pharmaceutically acceptable salt thereof and rituximab, administered without Compound A. In some embodiments, the improvement of clinical benefit rate is at least about 20%. In some embodiments, the improvement of clinical benefit rate is at least about 30%. In some embodiments, the improvement of clinical benefit rate is at least about 40%. In some embodiments, the improvement of clinical benefit rate is at least about 50%. In some embodiments, the improvement of clinical benefit rate is at least about 60%. In some embodiments, the improvement of clinical benefit rate is at least about 70%. In some the improvement of clinical benefit rate is at least about 80%.

### General Synthesis

Compounds of this invention can be made by the synthetic procedures described below. The starting materials and reagents used in preparing these compounds are either available from commercial suppliers such as Aldrich Chemical Co. (Milwaukee, Wis.), or Bachem (Torrance, Calif.), or are prepared by methods known to those skilled in the art following procedures set forth in references such as Fieser and Fieser's Reagents for Organic Synthesis, Volumes 1-17 (John Wiley and Sons, 1991); Rodd's Chemistry of Carbon Compounds, Volumes 1-5 and Supplementals (Elsevier Science Publishers, 1989); Organic Reactions, Volumes 1-40 (John Wiley and Sons, 1991), March's Advanced Organic Chemistry, (John Wiley and Sons, 4^{th} Edition) and Larock's Comprehensive Organic Transformations (VCH Publishers Inc., 1989). These schemes are merely illustrative of some methods by which the compounds of this invention can be synthesized, and various modifications to these schemes can be made and will be suggested to one skilled in the art having referred to this disclosure. The starting materials and the intermediates of the reaction may be isolated and purified if desired using conventional techniques, including but not limited to filtration, distillation, crystallization, chromatography and the like. Such materials may be characterized using conventional means, including physical constants and spectral data.

Unless specified to the contrary, the reactions described herein take place at atmospheric pressure and over a temperature range from about −78° C. to about 150° C., more specifically from about 0° C. to about 125° C. and more specifically at about room (or ambient) temperature, e.g., about 20° C. Unless otherwise stated (as in the case of an hydrogenation), all reactions are performed under an atmosphere of nitrogen.

Prodrugs can be prepared by techniques known to one skilled in the art. These techniques generally modify appropriate functional groups in a given compound. These modified functional groups regenerate original functional groups by routine manipulation or in vivo. Amides and esters of the compounds of the present invention may be prepared according to conventional methods. A thorough discussion of prodrugs is provided in T. Higuchi and V. Stella, “Pro-drugs as Novel Delivery Systems,” Vol 14 of the A.C.S. Symposium Series, and in Bioreversible Carriers in Drug Design, ed. Edward B. Roche, American Pharmaceutical Association and Pergamon Press, 1987, both of which are incorporated herein by reference for all purposes.

The compounds of the invention, or their pharmaceutically acceptable salts, may have asymmetric carbon atoms or quaternized nitrogen atoms in their structure. Compounds of Formula I that may be prepared through the syntheses described herein may exist as single stereoisomers, racemates, and as mixtures of enantiomers and diastereomers. The compounds may also exist as geometric isomers. All such single stereoisomers, racemates and mixtures thereof, and geometric isomers are intended to be within the scope of this invention. Some of the compounds of the invention may exist as tautomers. For example, where a ketone or aldehyde is present, the molecule may exist in the enol form; where an amide is present, the molecule may exist as the imidic acid; and where an enamine is present, the molecule may exist as an imine. All such tautomers are within the scope of the invention. In particular, imidazol-5-yl and pyrazol-5-yl each can also exist in their respective tautomeric forms imidazol-4-yl and pyrazol-3-yl. Regardless of which structure or which terminology is used, each tautomer is included within the scope of the Invention.

The present invention also includes N-oxide derivatives and protected derivatives of compounds of Formula I. For example, when compounds of Formula I contain an oxidizable nitrogen atom, the nitrogen atom can be converted to an N-oxide by methods well known in the art. When compounds of Formula I contain groups such as hydroxy, carboxy, thiol or any group containing a nitrogen atom(s), these groups can be protected with a suitable “protecting group” or “protective group”. A comprehensive list of suitable protective groups can be found in T. W. Greene, Protective Groups in Organic Synthesis, John Wiley & Sons, Inc. 1991, the disclosure of which is incorporated herein by reference in its entirety. The protected derivatives of compounds of Formula I can be prepared by methods well known in the art.

Methods for the preparation and/or separation and isolation of single stereoisomers from racemic mixtures or non-racemic mixtures of stereoisomers are well known in the art. For example, optically active (R)- and (S)-isomers may be prepared using chiral synthons or chiral reagents, or resolved using conventional techniques. Enantiomers (R- and S-isomers) may be resolved by methods known to one of ordinary skill in the art, for example by: formation of diastereoisomeric salts or complexes which may be separated, for example, by crystallization; via formation of diastereoisomeric derivatives which may be separated, for example, by crystallization, selective reaction of one enantiomer with an enantiomer-specific reagent, for example enzymatic oxidation or reduction, followed by separation of the modified and unmodified enantiomers; or gas-liquid or liquid chromatography in a chiral environment, for example on a chiral support, such as silica with a bound chiral ligand or in the presence of a chiral solvent. It will be appreciated that where a desired enantiomer is converted into another chemical entity by one of the separation procedures described above, a further step may be required to liberate the desired enantiomeric form. Alternatively, specific enantiomer may be synthesized by asymmetric synthesis using optically active reagents, substrates, catalysts or solvents or by converting on enantiomer to the other by asymmetric transformation. For a mixture of enantiomers, enriched in a particular enantiomer, the major component enantiomer may be further enriched (with concomitant loss in yield) by recrystallization.

In addition, the compounds of the present invention can exist in unsolvated as well as solvated forms with pharmaceutically acceptable solvents such as water, ethanol, and the like. In general, the solvated forms are considered equivalent to the unsolvated forms for the purposes of the present invention.

The chemistry for the preparation of the compounds of this invention is known to those skilled in the art. In fact, there may be more than one process to prepare the compounds of the invention. For specific examples, see M. Barvian et al. J. Med. Chem. 2000, 43, 4606-4616; S. N. VanderWei et al. J. Med. Chem. 2005, 48, 2371-2387; P. L. Toogood et al. J. Med. Chem. 2005, 48, 2388-2406; J. Kasparec et al. Tetrahedron Letters 2003, 44, 4567-4570; and references cited therein. See also U.S. Pre-grant publication US2004/0009993 A1 (M. Angiolini et al.), which is incorporated herein by reference, and references cited therein. The following examples illustrate but do not limit the invention. All references cited herein are incorporated by reference in their entirety.

A compound of the invention where R¹ is optionally substituted alkyl, R² is hydrogen or optionally substituted alkyl, R⁴ is methyl or ethyl, R⁶ is phenyl or heteroaryl each of which is optionally substituted with 1, 2, 3, 4, or 5 R⁹ groups (as defined in the Summary of the Invention), and R² is hydrogen can be prepared according to Scheme 1.

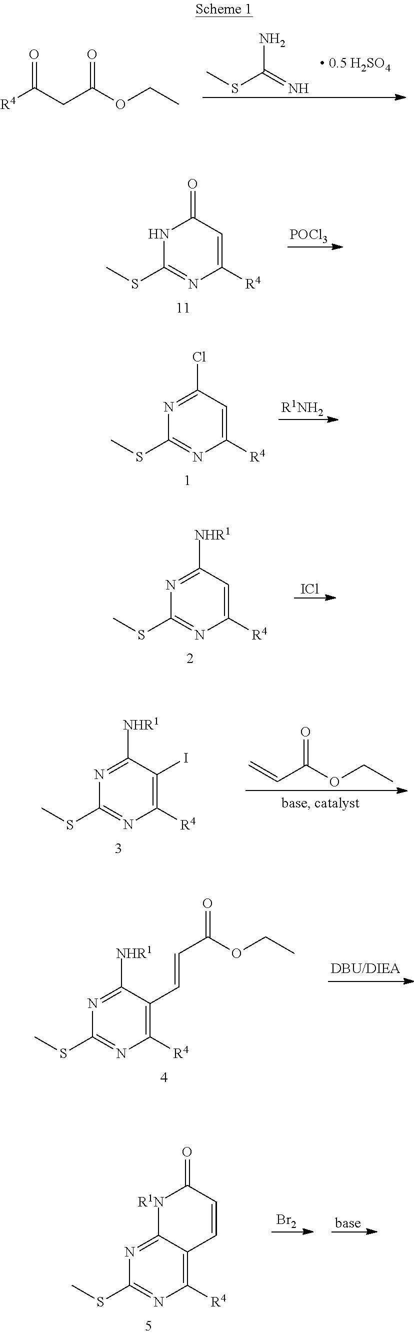

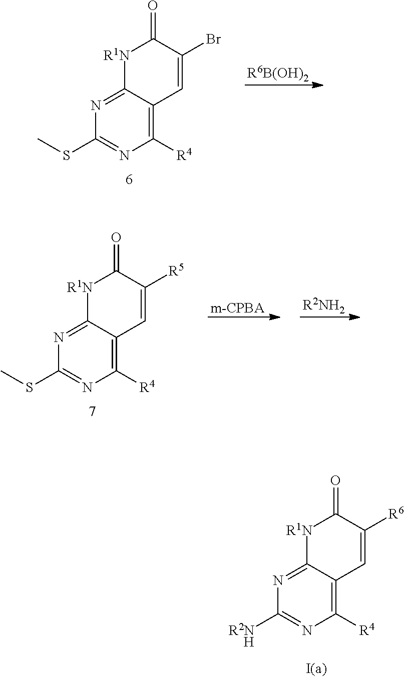

To a solution of commercially available 2-methyl-2-thiopseudourea sulfate in a solvent such as water is added a base such as sodium carbonate and an intermediate of formula 10 at room temperature. The reaction mixture is stirred for overnight or less. After neutralizing, intermediate 11 is collected through filtration and followed by drying under vacuum. Intermediate 11 is then treated with POCl₃ and the reaction is heated to reflux for approximately 2 h and then concentrated under vacuum to dryness. Intermediate 1 can be used directly in the next-reaction without further purification.

An intermediate of formula 2 is prepared by reacting an intermediate of formula 1 with a primary amine R¹NH₂ in a solvent such as water and with heating. Intermediate 2 is then treated with iodine monochloride in a solvent such as methanol at around 0° C. and allowed to react for approximately overnight or less as needed for the reaction to go to completion to form intermediate 3. After completion the residue is triturated with acetone. The intermediate 3 is then reacted in a solvent, such as DMA, with ethyl acrylate in the presence of a base, such as triethylamine, and in the presence of a catalyst, such as Pd(OAc)₂, and (+)BINAP. The reaction is heated to approximately 100° C. and allowed to react for approximately overnight or less as needed for the reaction to go to completion to form intermediate 4. Intermediate 4 is then optionally purified by column chromatography.

Intermediate 5 is prepared by treating intermediate 4 with DBU in the presence of a base such as DIPEA at room temperature. The reaction mixture is then heated to reflux and reacted for approximately 15 h. After evaporation of solvent, the residue is triturated with acetone and collected by filtration to yield 5.

Intermediate 6 is prepared by reacting intermediate 5 with a brominating agent such as Br₂ in a solvent such as DCM at room temperature. The reaction mixture is then stirred for approximately overnight. The resulting product is filtered and then suspended in a solvent such as DCM and treated with a base such as triethylamine. The mixture is then washed with water and dried over a drying agent such as Na₂SO₄ to yield intermediate 6.

A Suzuki coupling is then performed using intermediate 6 and a boronic acid (or ester) of formula R⁶B(OH)₂ in a solvent(s) such as a DME-H₂O mixture in the presence of a catalyst such as Pd(dpppf) and a base such as triethylamine at room temperature. The reaction mixture is heated to reflux for approximately 4 h. After cooling to room temperature, the reaction mixture is partitioned with water and ethyl acetate. After separation, the organic layer is dried over a drying agent such as Na₂SO₄ to yield intermediate 7.

The methylthio group of intermediate 7 is then oxidized with m-CPBA in a solvent such as DCM at room temperature allowing to stir for approximately 4 h. After removal of the solvent under reduced pressure, the product is treated with an amine of formula R²NH₂ in a solvent such as dioxane and stirred at room temperature for approximately overnight to yield a Compound of Formula I.

Alternatively, a Compound of Formula I where R¹ is optionally substituted alkyl, R⁴ is methyl or ethyl, R⁶ is phenyl or heteroaryl each of which is optionally substituted with 2, 3, 4, or 5 R⁹ groups (as defined in the Summary of the Invention), and R² is hydrogen can be prepared according to Scheme 2.

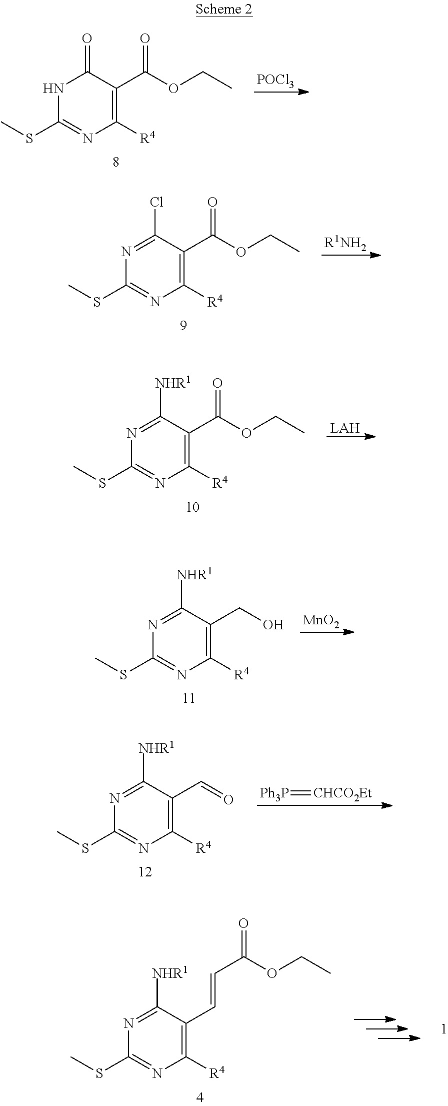

An intermediate of formula 9 is prepared by reacting an intermediate of formula 8 with neat POCl₃ and heating. 9 is then treated with a primary amine R¹NH₂ in a solvent such as water or THF and triethylamine at 0° C. to form 10. After removal of the solvent under reduced pressure, the intermediate 10 is then reacted with lithium aluminum hydride in a solvent such as THF at 0° C. After quenching and aqueous workup, solvent removal provided crystalline 11 without further purification. Treatment of 11 with manganese (II) dioxide in a solvent such as methylene chloride or chloroform at room temperature provided aldehyde 12 upon filtration and solvent removal. A Wittig reaction with aldehyde 12 can be employed with (carbethoxymethylene)triphenylphosphorane in refluxing THF to provides the common intermediate 4. 4 can then be used to prepare a Compound of Formula I using the procedures described in Scheme 1.

A compound of the invention where R¹ is optionally substituted alkyl, R⁴ is methyl or ethyl, R⁶ is phenyl or heteroaryl each of which is optionally substituted with 1, 2, 3, 4, or 5 R⁹ groups as defined in the Summary of the Invention), and R² is hydrogen can be prepared according to Scheme 3.

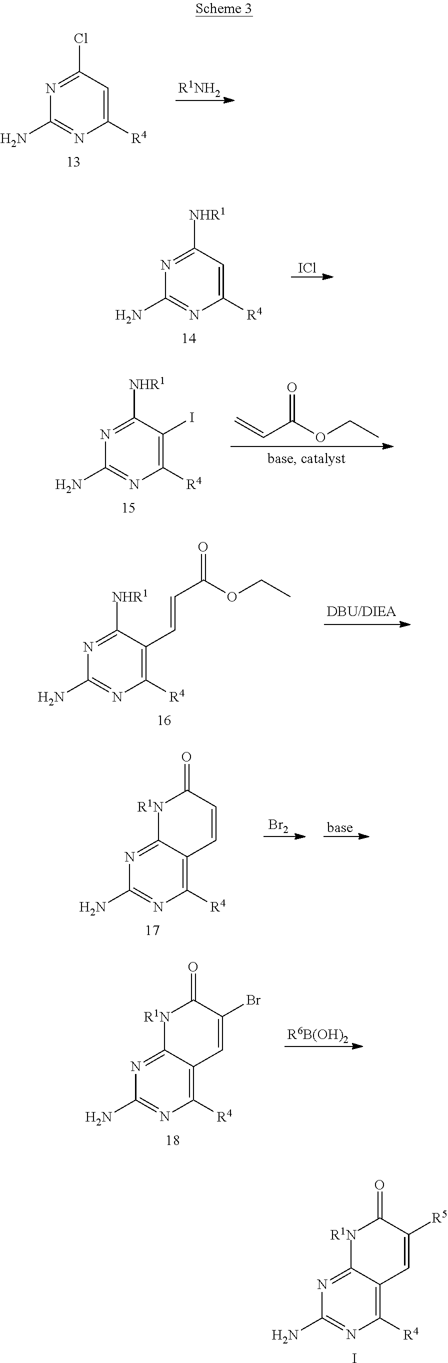

An intermediate of formula 14 is prepared by reacting an intermediate of formula 13 with a primary amine R¹NH₂ in a solvent such as water and with heating. 14 is then treated with iodine monochloride in a solvent such as methanol at around 0° C. and allowed to react for approximately overnight or less as needed for the reaction to go to completion to form 15. After completion the residue is triturated with acetone. The intermediate 15 is then reacted in a solvent, such as DMA, with ethyl acrylate in the presence of a base, such as triethylamine, and in the presence of a catalyst, such as Pd(OAc)₂, and (+)BINAP. The reaction is heated to approximately 100° C. and allowed to react for approximately overnight or less as needed for the reaction to go to completion to form 16. 16 is then optionally purified by column chromatography. A Compound of Formula I can then be prepared from 16 by using the same reaction conditions as described in Scheme 1 (starting at the point of the preparation of 5 from 4).

A compound of the invention where R¹ is optionally substituted alkyl, R⁴ is methyl or ethyl, R⁶ is phenyl or heteroaryl each of which is optionally substituted with 1, 2, 3, 4, or 5 R⁹ groups (as defined in the Summary of the Invention), and R² is hydrogen can alternatively be prepared according to Scheme 4.

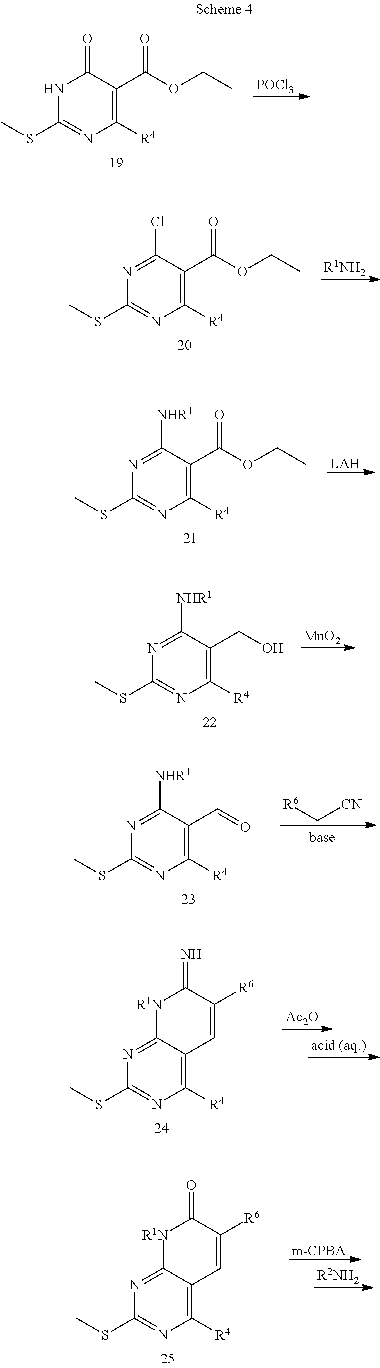

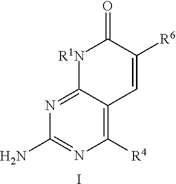

An intermediate of formula 20 is prepared by reacting an intermediate of formula 19 with neat POCl₃ and heating. 20 is then treated with a primary amine R¹NH₂ in a solvent such as water or THF and triethylamine at 0° C. to form 21. After removal of the solvent under reduced pressure, the intermediate 21 is then reacted with lithium aluminum hydride in a solvent such as THF at 0° C. After quenching and aqueous workup, solvent removal provides crystalline 22 without further purification. Treatment of 22 with manganese (II) dioxide in a solvent such as methylene chloride or chloroform at room temperature provides aldehyde 23 upon filtration and solvent removal. A Knovenegal-type condensation with 23 and an arylacetonitrile in the presence of a base such as potassium carbonate or sodium hydroxide in a protic solvent provides the cyclized imine 24. Acetylation of the imine with acetic anhydride is required prior to hydrolysis, which takes place in the presence of aqueous acid and heating to afford 25. Subsequently, 25 can be oxidized to the corresponding sulfone with m-CPBA at room temperature and displaced with ammonium to provide I.

The synthesis of specific compounds is described in WO2007 0444813 which is hereby incorporated by reference in its entirety.

### EXAMPLES

### Example 1

### In Vivo Evaluation of Compound A in Combination with Bendamustine

To evaluate the antitumor activity of Bendamustine in combination with the dual pan-PI3K/mTOR inhibitor Compound A, experiments were conducted using female SCID mice bearing human WSU-DLCL2 xenografts. In this study, Bendamustine at 3.5 and 7 mg/kg was tested in combination with Compound A at 20 mg/kg.

### Materials and Methods

CB17/1CR-Prkdc severe combined immunodeficiency (SCID)/Crl mice, at 8-10 weeks old, were bred at Charles River France (Domaine des Oncins, 69210 L'Arbreste, France) from strains obtained from Charles River, USA. Mice were over 18 g at start of treatment after an acclimatization time of at least 5 days. They had free access to food (UAR reference 113, Villemoisson, 91160 Epinay sur Orge, France) and sterile water. They were housed on a 12 hours light/dark cycle. Environmental conditions including animal maintenance, room temperature (22° C.±2° C.), relative humidity (55%±15%) and lighting times were recorded by the supervisor of laboratory animal sciences and welfare (LAW) and the records are archived.

The human WSU-DLCL2 tumor model was established by implanting (SC) small tumor fragments and was maintained in SCID female mice using serial passages.

Bendamustine formulation was prepared by incorporating the compound into NaCl 0.9% at pH 3. The preparation was prepared daily prior administration. The volume of IP administration per mouse was 10 mL/kg.

Compound A formulations were prepared in 1N HCl and water for injection, final pH was 3, followed by five cycles of vortexing and sonicating. The stock solution was chemically stable 7 days in the dark at 4° C. The volume of PO administration per mouse was 10 mL/kg.

For subcutaneous implantation of tumor cells, skin in the flank of the mice was disinfected using alcohol or Betadine® solution (Alcyon) and a suspension of tumor cells was inoculated SC unilaterally under a volume of 0.2 mL using a 23 G needle.

The dosages and schedule of administration of Bendamustine and Compound A used as single agent or in combination are described in the results section and detailed in the tables that follow.

The animals required to begin a given experiment were pooled and implanted monolaterally on day 0. Treatments were administered on measurable tumors. The solid tumors were allowed to grow to the desired volume range (animals with tumors not in the desired range were excluded). The mice were then pooled and unselectively distributed to the various treatment and control groups. Treatment started 20 days post WSU-DLCL2 tumor fragment implantation as indicated in the results section and in each table. The dosages are expressed in mg/kg, based on the body weight at start of therapy. Mice were checked daily, and adverse clinical reactions noted. Each group of mice was weighed as a whole daily until the weight nadir was reached. Then, groups were weighed once to thrice weekly until the end of the experiment. Tumors were measured with a calliper 2 to 3 times weekly until final sacrifice for sampling time, tumor reached 2000 mm³ or until the animal died (whichever comes first). Solid tumor volumes were estimated from two-dimensional tumor measurements and calculated according to the following equation:

Tumor weight (mg)=Length (mm)×Width²(mm²)/2

The day of death was recorded. Surviving animals were sacrificed and macroscopic examination of the thoracic and abdominal cavities was performed.

A dosage producing a 15% body weight loss (BWL) during three consecutive days (mean of group), 20% BWL during 1 day or 10% or more drug deaths was considered an excessively toxic dosage. Animal body weights included the tumor weight.

The primary efficacy end points are ΔT/ΔC, percent median regression, partial and complete regressions (PR and CR).

Changes in tumor volume for each treated (T) and control (C) group were calculated for each tumor by subtracting the tumor volume on the day of first treatment (staging day) from the tumor volume on the specified observation day. The median ΔT is calculated for the treated group, and the median ΔC is calculated for the control group. Then the ratio ΔT/ΔC is calculated and expressed as a percentage. The dose is considered as therapeutically active when ΔT/ΔC is lower than 40% and very active when ΔT/ΔC is lower than 10%. If ΔT/ΔC is equal to or lower than 0, the dose is considered as highly active.

Partial regression: Regressions are defined as partial if the tumor volume decreases to 50% or the tumor volume at the start of treatment.

Complete regression: The CR is achieved when tumor volume=0 mm³ (CR is considered when tumor volume cannot be recorded).

The term “therapeutic synergy” is used when the combination of two products at given doses is more efficacious than the best of the two products alone considering the same doses. In order to study therapeutic synergy, each combination was compared to the best single agent using estimates obtained from a two-way analysis of variance with repeated measurements (Time factor) on parameter tumor volume.

Statistical analyses were performed on SAS system release 8.2 for SUN4 via Everstat V5 software and SAS 9.2 software. A probability less than 5% (p<0.05) was considered as significant.

### Results

The median tumor burden at start of therapy was 150 to 176 mm³. As single agents, Compound A (20 mg/kg/adm) was administered daily from days 20 to 31 post tumor implantation, and Bendamustine (3.5 and 7 mg/kg/adm) was administered daily from days 20 to 25. In the combination groups, the doses of Compound A and Bendamustine were administered as shown in Table 1.

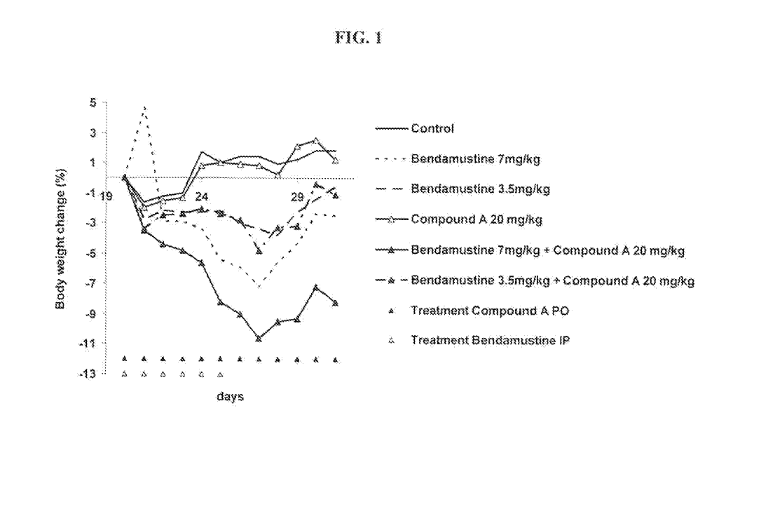

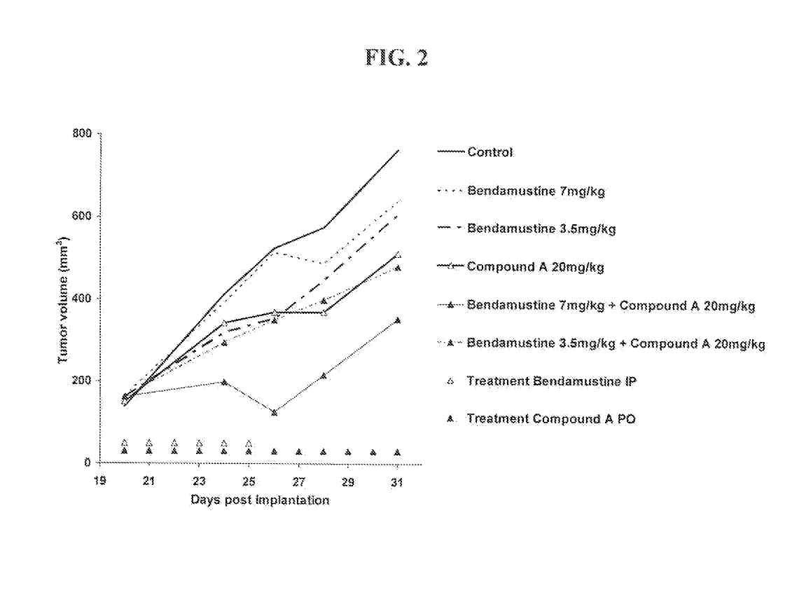

Compound A and Bendamustine as Single agents or used in combination were tolerated (Table 2 and FIG. 1). In Table 2, one mouse was excluded from the study on days 28 and 31 (due to unreliable tumor measurement). As single agents, Bendamustine (3.5 and 7 mg/kg) and Compound A were inactive (ΔT/ΔC>40) (Table 1 and FIG. 2) under these conditions. In combination, treatment with Bendamustinc at 7 mg/kg and Compound A was active (ΔT/ΔC=33 on day 31) and induced 1 PR and as shown by Table 2, therapeutic synergy was reached (p=0.0018 for global analysis). See also Table 3.

TABLE 2
Antitumor activity Compound A in combination with Bendamustine against human WSU-DLCL2 bearing SCID female mice

| | | Dosage in | | | Average body weight | | | |
|---|---|---|---|---|---|---|---|---|
| | Route/Dosage | mg/kg per | | Drug death | change in % per | Median | | |
| Agent | in mL/kg | injection | Schedule | (Day of | mouse at nadir | ΔT/ΔC in | Regressions | |
| (batch) | per injection | (total dose) | in days | death) | (day of nadir) | % day 31 | Partial | Complete |
| Bendamustine | IP 10 mL/kg | 7 (42) | 20-25 | 0/7 | −7.2 (27) | 82 | 0/7 | 0/7 |
| (VAC.FGU7. | | 3.5 (21) | | 0/7 | −3.8 (28) | 83 | 0/7 | 0/7 |
| 062) | | | | | | | | |
| Compound A | PO 10 mL/kg | 20 (240) | 20-31 | 0/7 | −2.0 (21) | 54 | 0/7 | 0/7 |
| (T1007388) | | | | | | | | |
| Bendamustine | IP 10 mL/kg | 7 (42) | 20-25 | 0/7 | −10.6 (27) | 33 | 1/7 | 0/7 |
| Compound A | PO 10 mL/kg | 20 (240) | 20-31 | | | | | |
| | | 3.5 (21) | | 0/7 | −4.8 (25) | 55 | 0/7 | 0/7 |
| | | 20 (240) | | | | | | |
| Control * | | | | | −1.6 (21) | | 0/7 | 0/7 |

Tumor size at start of therapy was 80-270 mm³, with a median tumor burden per group of 150-176 mm³. Drug formulation for Bendamustine consisted of NaCl 0.9% in Water, pH 3; Compound A consisted of water, pH 3. Treatment duration for Bendamustine was 6 days; the treatment duration for Compound A was 12 days. The abbreviations used were ΔT/ΔC for ratio of change in tumor volume from baseline median between treated and control groups (TVday−TV0)/(CVdav−CV0)*100.

TABLE 3
Antitumor activity of Compound A in combination with Bendamustine against human
WSU-DLCL2 bearing SCID female mice: Therapeutic synergy determination.
Median (nMad) and Anova followed by a Dunnett's
test on rank-transformed tumor volume changes from baseline

| | | Day | | | | |
|---|---|---|---|---|---|---|
| Group | | Global | 24 | 26 | 28 | 31 |
| Bendamustine 7 | mg/kg + | — | 19 (10.4) | −41 (96.4) | 0 (80.1) | 190 (97.9) |
| Compound A 20 | mg/kg | | n = 7 | n = 7 | n = 7 | n = 7 |
| | | — | — | — | — | — |
| Bendamustine 7 | mg/kg | — | 267 (198.7) | 317 (258) | 306 (268.4) | 477 (249.1) |
| | | | n = 7 | n = 7 | n = 7 | n = 7 |
| | | p = 0.0001 | p = 0.0030 | p = 0.0004 | p = 0.0011 | p = 0.0072 |
| Compound A 20 | mg/kg | — | 145 (139.4) | 218 (26.7) | 242 (172) | 314 (317.3) |
| | | | n = 7 | n = 7 | n = 7 | n = 7 |
| | | p = 0.0018 | p = 0.0886 | p = 0.0042 | p = 0.0024 | p = 0.0248 |
| Bendamustine 3.5 | mg/kg + | — | 108 (127.5) | 132 (123.1) | 251 (108.2) | 318 (160.1) |
| Compound A 20 | mg/kg | | n = 7 | n = 7 | n = 7 | n = 7 |
| | | — | — | — | — | — |
| Bendamustine 3.5 | mg/kg | — | 170 (62.3) | 222 (89) | 268 (252) | 483 (247.6) |
| | | | n = 7 | n = 7 | n = 7 | n = 7 |
| | | p = 0.4087 | p = 0.1972 | p = 0.6910 | p = 0.9990 | p = 0.3448 |
| Compound A 20 | mg/kg | — | 145 (139.4) | 218 (26.7) | 242 (172) | 314 (317.3) |
| | | | n = 7 | n = 7 | n = 7 | n = 7 |
| | | p = 0.9395 | p = 0.6373 | p = 0.7242 | p = 0.8367 | p = 0.9973 |

p-value: obtained with a Dunnett's test versus combination Bendamustine Compound A after 2-way Anova with repeated measures on rank-transformed tumor volume changes from baseline

TABLE 4

| | | □T/□C (%) on | |
|---|---|---|---|
| | | d31 | |
| Bendamustine 7 | mg/kg | 82 | |
| Bendamustine 3.5 | mg/kg | 83 | |
| Compound A 20 | mg/kg | 54 | |
| Bendamustine 7 | mg/kg | 33 | p = 0.0018 vs. |
| Compound A 20 | mg/kg | | Compound A |
| | | | 20 mg/kg |
| Bendamustine 3.5 | mg/kg | 55 | |
| Compound A 20 | mg/kg | | |

### Example 2

### Treatment of Hematological Malignancies with Compound A

A Phase 1b, multicenter, open-label, dose-escalation study of Compound A is conducted to evaluate the safety, tolerability, and clinical activity of Compound A in combination with bendamustine and/or rituximab in patients with relapsed or refractory indolent B-cell non-Hodgkin lymphoma (iNHL), mantle cell lymphoma (MCL), or chronic lymphocytic leukemia (CLL).

### Study Objectives

The primary objective of the study is to determine the maximum tolerated dose (MTD) and recommended Phase 2 dose for Compound A when administered in combination with bendamustine and/or rituximab. The secondary objectives include:
To determine the safety and tolerability of Compound A in combination with bendamustine and/or rituximab in patients with indolent non-Hodgkin lymphoma (iNHL) mantle cell lymphoma (MCL) or chronic lymphocytic leukemia (CLL)
To determine the pharmacokinetics (PK) of Compound A, bendamustine and rituximab when used in combination in patients with iNHL, MCL, or CLL
To determine the pharmacodynamic (PD) effects of Compound A in combination with bendamustine and/or rituximab in patients with iNHL, MCL, or CLL
To determine the antitumor activity of Compound A in combination with bendamustine and/or rituximab in patients with iNHL, MCL, or CLL

An exploratory objective is to explore correlations between preexisting molecular alterations directly or indirectly involved in PI3K/mTOR and other pathway(s) and response and/or resistance to Compound A in combination with bendamustine and/or rituximab in patients with iNHL, MCL, or CLL.

### Study Design

This is a Phase 1b trial design. It will be conducted as a 3-arm, dose-escalation, nonrandomized, open-label, multi-institutional study.

### Dose Escalation Phase

Arm A will receive fixed doses of rituximab and increasing doses of Compound A.

Arms B1 and B2 will receive fixed doses of bendamustine and rituximab and increasing doses of Compound A. All patients will require inpatient admission for Cycle 1, Days 1 and Day 2 to ensure patient safety and adequate PK and PD sampling. Arm A will enroll patients with relapsed/refractory iNHL, MCL, or CLL. Arm B1 will enroll patients with relapsed/refractory iNHL or MCL. Arm B2 will enroll patients with relapsed/refractory CLL.

Each cohort will enroll 3 to 6 patients at each dosing level. Arms A, B1 and B2 will enroll simultaneously. The starting dose of Compound A will be 30 mg twice daily (BID) with escalation to 50 mg BID (ie, MTD of Compound A administered as single agent). An intermediate Compound A dose level (ie, 40 mg BID) or a lower dose level (<30 mg bid) may be tested based on dose-limiting toxicity (DLT) observation.

### Dose Levels

Arm A: Compound A and rituximab: iNHL, MCL, or CLL

Dose level 1: rituximab 375 mg/m2; Compound A 30 mg BID
Dose level 2: rituximab 375 mg/m2; Compound A 50 mg BID

Arm B1: Compound A, bendamustine, rituximab: iNHL or MCL

Dose level 1: rituximab 375 mg/m2; bendamustine 90 mg/m2; Compound. A 30 mg BID
Dose level 2: rituximab 375 mg/m2; bendamustine 90 mg/m2; Compound A 50 mg BID

Arm B2: Compound A, bendamustine, rituximab: CLL

Dose level 1: rituximab 375 mg/m2; bendamustine 70 mg/m2; Compound A 30 mg BID
Dose level 2: rituximab 375 mg/m2; bendamustine 70 mg/m2; Compound A 50 mg BID

### Maximum Tolerated Dose Expansion Phase

The MID is defined as the highest dose level at which no more than 1 patient of a maximum of 6 pts experienced an investigational medicinal product (IMP)-related DLT. In each arm and tumor-type cohort, a minimum of 6 additional patients will be treated at the preliminary MTD to further evaluate the safety and tolerability of this dose in combination with rituximab or bendamustin plus rituximab. A maximum of 12 patients will be treated at the MTD close level per arm and per tumor type. The recommended Phase 2 dose of Compound A will be determined for each treatment arm (A, B1, B2).

### Main Inclusion Criteria

Histologically or cytologically and phenotypically confirmed diagnosis of NHL, MCL or CLL who have relapsed or have been refractory to at least 1 standard therapy. Refractory disease is defined as unresponsive to a standard regimen or progressing within 6 months of completing a standard regimen.

The total expected number of patients is approximately 18 to 36 patients for the dose escalation phase and approximately 18 to 27 patients (6 to 9 per arm) for the maximum tolerated dose expansion cohorts.

### Routes of Administration

Compound A is administered orally and is supplied as capsule formulations at strengths of 10-, 30-, 40-, and 50-mg capsules for oral administration. Rituximab is administered as an intravenous (IV) infusion (3 to 4 hour infusion, refer to package insert). Bendamustine also is administered as an IV infusion (30 to 60 minute infusion, refer to package insert).

### Dose Regimen/Duration

All patients will take Compound A BID (in the morning and evening), with a preferred interval of 12 (±1) hours between doses. Compound A should be taken with 1 glass (approximately 8 ounces (240 mL) of water, with no food allowed for at least 2 hours before and 1 hour after dosing. If a dose is missed it may be taken up to 4 hours after the normal dosing time. No doses outside the 4-hour window should be given or made up at a future time. Extra doses should not be administered if the patient vomits after taking IMP. Patients may take other concomitant medications (except gastric pH-altering medications) with water at the same time that Compound A is administered.

Combination therapy with Compound A and rituximab as doublet therapy, or with bendamustine and rituximab as triplet therapy, will be administered over a 28-day cycle. The starting dose for Compound A will be 30 mg BID.

### Arm A

All patients will receive Compound A orally BID as long as there is clinical benefit
Patients will receive rituximab 375 mg/m2 IV weekly (Days 1, 8, 15, and 22 of a 28-day cycle) for 2 cycles.

### Arm B1

Patients with MCL/iNHL will receive rituximab 375 mg/m2 IV on Day 1 and will receive bendamustine 90 mg/m2 IV on Days 1 and 2 of each 28-day cycle for up to 8 cycles. Discontinuation of rituximab after completing Cycle 2 may be permitted at the discretion of the Investigator after discussion with the Sponsor.

### Arm B2

Patients with CLL will receive rituximab 375 mg/m2 on Cycle 1, Day 1, then 500 mg/m2 on Day 1 of Cycles 2 through 6 and will receive bendamustine 70 mg/m2 IV on Days 1 and 2 of each 28-day cycle up to 6 cycles. Discontinuation of rituximab after completing Cycle 2 may be permitted at the discretion of the Investigator after discussion with the Sponsor.

All patients in Arms B1 and B2 must receive tumor lysis syndrome (TLS) prophylaxis with oral allopurinol during cycle 1 (2 days prior and 5 days after C1D1) and must be admitted to hospital for Cycle 1 Days 1 and Day 2 for hydration and monitoring.

### Primary Endpoints

### Dose-Limiting Toxicity

Dose-limiting toxicity will be defined as any 1 of the following toxicities occurring during Cycle 1 of the study treatment using the current National Cancer Institute (NCI) Common Terminology Criteria for Adverse Events (CTCAE) criteria (v4.03):

### Hematologic Toxicity

Grade 4 neutropenia (ANC<0.5×10⁹/L) lasting >7 days.
Febrile neutropenia defined as an ANC of <1.0×10⁹/L with a single temperature >38.3° C. or a sustained temperature ≧38° C. for more than 1 hour.
Grade 4 thrombocytopenia (platelet count <25.0×10⁹/L) lasting >7 days or of any duration associated with Grade>3 hemorrhage.

### Nonhematologic Toxicity

Any Grade≧3 nonhematologic toxicity except diarrhea, nausea, or vomiting
   Nausea/vomiting or diarrhea will be considered a DLT in patients who have Grade≧3 toxicity for ≧2 days despite receiving optimal prophylaxis and/or treatment
   Grade 4 diarrhea of any duration will be considered a DLT
Any Grade 3 rash in patients receiving Compound A in combination with bendamustine failing to recover to Grade≦1 toxicity by Day 28
Any toxicity resulting in a treatment delay >2 weeks
A treatment-emergent adverse event (TEAE) that in the opinion of the study committee is of potential clinical significance such that further dose escalation would expose patients to unacceptable risk

### Maximum Tolerated Dose

The MTD is the largest dose level at which at most 1 patient of a dose-level cohort of 6 patients (or <33% of patients at a dose level with more patients) experiences study treatment-related DLT.

### Secondary Endpoints

### Safety

The number and proportion of patients experiencing TEAEs. The number and proportion of patients experiencing clinically significant changes in a laboratory parameter and/or vital signs.

### Pharmacokinetic Parameters

Plasma pharmacokinetic (PK) parameters for Compound A, AUC0-12 h, Cmax and tmax will be assessed for the morning dose on Day 1 of Cycles 1 and 2. Bendamustine and its M3 metabolite PK parameters. AUC, AUClast, Ceoi, tmax, Cl and Vss will be assessed on Day 1 of Cycles 1 and 2. Rituximab PK parameters, AUC0-7 h, Ceoi and tmax, will be assessed on Day 1 of Cycles 1 and 2.

### Efficacy

Objective response rate (ORR) will be assessed to determine antitumor activity. Objective response rate is defined as the proportion of patients who experience complete response/remission (CR) or partial response/remission (PR) as defined by the International Working Group (IWG) response criteria for malignant lymphoma (1) and for CLL (2).

### Pharmacodynamics

Molecular pathway modulation as determined by change of mechanistic markers pAKT) will be determined using pre- and postdose blood, and tumor tissues. Markers of proliferation and apoptosis will also be measured. Sampling details are provided in flow charts (Section 1.3).

### Exploratory Endpoints

Molecular characterization of the most recent archival or fresh tumor tissues/cells and baseline blood samples will be performed when technically feasible to explore correlation of preexisting molecular alterations in PI3K/mTOR and/or other pathway(s) and treatment outcome.

The foregoing invention has been described in some detail by way of illustration and example, for purposes of clarity and understanding. The invention has been described with reference to various specific embodiments and techniques. However, it should be understood that many variations and modifications may be made while remaining within the spirit and scope of the invention. It will be obvious to one of skill in the art that changes and modifications may be practiced within the scope of the appended claims. Therefore, it is to be understood that the above description is intended to be illustrative and not restrictive. The scope of the invention should, therefore, be determined not with reference to the above description, but should instead be determined with reference to the following appended claims, along, with the full scope of equivalents to which such claims are entitled. All patents, patent applications and publications cited in this application are hereby incorporated by reference in their entirety for all purposes to the same extent as if each individual patent, patent application or publication were so individually denoted.

1. A method of treating cancer in a patient, comprising administering to the patient an effective amount of (a) a Compound of Formula IA: 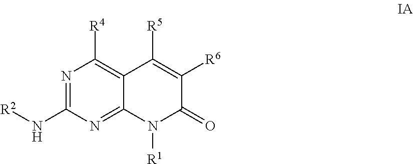
or a metabolite or a pharmaceutically acceptable salt thereof; and either (b) bendamustine or (c) rituximab or (d) a combination of bendamustine and cituximab,
wherein for the Compound of Formula IA:
R¹ is alkyl, cycloalkyl, cycloalkylalkyl, aryl, arylalkyl, heterocycloalkyl, heterocycloalkylalkyl, heteroaryl or heteroarylalkyl;
R² is hydrogen or alkyl;
R⁴ is alkyl;
R⁵ is hydrogen; and
R⁶ is phenyl, acyl, or heteroaryl wherein the phenyl and heteroaryl are is optionally substituted with 1, 2, 3, 4, or 5 R⁹ groups; and
each R⁹, when present, is independently halo, alkyl, haloalkyl, alkoxy, haloalkoxy, cyano, amino, alkylamino, dialkylamino, alkoxyalkyl, carboxyalkyl, alkoxycarbonyl, aminoalkyl, cycloalkyl, aryl, arylalkyl, aryloxy, heterocycloalkyl, or heteroaryl and where the cycloalkyl, aryl, heterocycloalkyl, and heteroaryl, each either alone or as part of another group within R⁹, are independently optionally substituted with 1, 2, 3, or 4 groups selected from halo, alkyl, haloalkyl, hydroxy, alkoxy, haloalkoxy, amino, alkylamino, and dialkylamino.

2. The method of claim 1, wherein R¹ in the compound of formula IA is alkyl, cycloalkyl, heterocycloalkylalkyl, or arylalkyl; R₂ is hydrogen or alkyl; R⁴ is alkyl; R⁵ is hydrogen; R⁶ is phenyl or heteroaryl wherein the phenyl and heteroaryl are is optionally substituted with one, two, or three R⁹ groups; each R⁸, when present, is independently amino, alkylamino, dialkylamino, or halo; and each R⁸, when present, is independently alkyl, arylalkyl, cyano, aryl, alkoxycarbonyl.

3. The method of claim 1, wherein R⁴ in the compound of formula IA is methyl.

4. The method of claim 1, wherein R¹ in the compound of formula IA is alkyl, cycloalkyl, or heterocycloalkyl.

5. The method of claim 1, wherein R¹ in the compound of formula IA is alkyl.

6. The method of claim 1, wherein R⁶ in the compound of formula IA is heteroaryl optionally substituted with 1, 2, or 3 R⁹ groups.

7. The method of claim 1, wherein R⁶ in the compound of formula IA is pyrazolyl, imidazolyl, thienyl, thiazolyl, oxazolyl, isoxazolyl, oxadiazolyl, furanyl, pyrrolyl, triazolyl, or tetrazolyl; each of which is optionally substituted with 1, 2, or 3 R⁹ groups.

8. The method of claim 1, wherein R⁶ in the compound of formula IA is pyrazol-3-yl, pyrazol-4-yl, pyrazol-5-yl, imidazol-2-yl, imidazol-4-yl, imidazol-5-yl, thien-2-yl, thien-3-yl, thiazol-2-yl, thiazol-4-yl, thiazol-5-yl, oxazol-2-yl, oxazol-4-yl, oxazol-5-yl, isoxazol-3-yl, isoxazol-4-yl, isoxazol-5-yl, 1,2,3-oxadiazol-4-yl, 1,2,3-oxadiazol-5-yl, 1,3,4-oxadiazol-2-yl, 1,2,4-oxadiazol-3-yl, 1,2,4-oxadiazol-5-yl, furan-2-yl, furan-3-yl, pyrrol-2-yl, pyrrol-3-yl, triazol-4-yl, triazol-5-yl, or tetrazol-5-yl; each of which is optionally substituted with 1, 2, or 3 R⁹ groups.

9. The method of claim 1, wherein R² in the compound of formula IA is hydrogen, R⁴ is methyl, R¹ is optionally substituted alkyl, cycloalkyl, or heterocycloalkyl, and R⁶ is heteroaryl optionally substituted with 1, 2, or 3 R⁹ groups.

10. The method of claim 1 wherein the compound of formula IA is selected from:
2-amino-8-ethyl-4-methyl-6-(1H-pyrazol-5-
yl)pyrido[2,3-d]pyrimidin-7(8H)-one;
2-amino-8-cyclopentyl-4-methyl-6-(1H-pyrazol-3-
yl)pyrido[2,3-d]pyrimidin-7(8H)-one;
2-amino-4-methyl-8-(1-methylethyl)-6-(1H-pyrazol-3-
yl)pyrido[2,3-d]pyrimidin-7(8H)-one;
2-amino-4-methyl-8-(phenylmethyl)-6-(1H-pyrazol-3-
yl)pyrido[2,3-d]pyrimidin-7(8H)-one;
2-amino-8-ethyl-4-methyl-6-(4-methyl-3-
thienyl)pyrido[2,3-d]pyrimidin-7(8H)-one;
2-amino-8-ethyl-4-methyl-6-(2-thienyl)pyrido[2,3-
d]pyrimidin-7(8H)-one;
2-amino-8-ethyl-4-methyl-6-(3-thienyl)pyrido[2,3-
d]pyrimidin-7(8H)-one;
2-amino-8-ethyl-6-furan-3-yl-4-methylpyrido[2,3-
d]pyrimidin-7(8H)-one;
2-amino-8-ethyl-4-methyl-6-phenylpyrido[2,3-
d]pyrimidin-7(8H)-one;
2-amino-8-ethyl-6-isoxazol-4-yl-4-methylpyrido[2,3-
d]pyrimidin-7(8H)-one;
2-amino-8-ethyl-6-furan-2-yl-4-methylpyrido[2,3-
d]pyrimidin-7(8H)-one;
5-(2-amino-8-ethyl-4-methyl-7-oxo-7,8-dihydropyrido[2,3-
d]pyrimidin-6-yl)thiophene-2-carbonitrile;
2-amino-8-ethyl-4-methyl-6-pyrimidin-5-ylpyrido[2,3-
d]pyrimidin-7(8H)-one;
2-amino-8-ethyl-6-(1H-imidazol-5-yl)-4-
methylpyrido[2,3-d]pyrimidin-7(8H)-one;
2-amino-8-ethyl-4-methyl-6-(1H-1,2,3-triazol-5-
yl)pyrido[2,3-d]pyrimidin-7(8H)-one;
2-amino-8-ethyl-4-methyl-6-(1H-pyrazol-4-
yl)pyrido[2,3-d]pyrimidin-7(8H)-one;
2-amino-8-ethyl-4-methyl-6-(1,3-thiazol-2-
yl)pyrido[2,3-d]pyrimidin-7(8H)-one;
2-amino-8-ethyl-4-methyl-6-(1H-tetrazol-5-
yl)pyrido[2,3-d]pyrimidin-7(8H)-one;
2-amino-8-ethyl-4-methyl-6-(1-methyl-1H-pyrrol-2-
yl)pyrido[2,3-d]pyrimidin-7(8H)-one;
2-amino-4,8-diethyl-6-(1H-pyrazol-5-
yl)pyrido[2,3-d]pyrimidin-7(8H)-one; and
2-amino-8-cyclopentyl-4-methyl-6-(1,3-thiazol-5-
yl)pyrido[2,3-d]pyrimidin-7(8H)-one.

11. The method of claim 10, wherein the compound of Formula IA is 2-amino-8-ethyl-4-methyl-6-(1H-pyrazol-5-yl)pyrido[2,3-d]pyrimidin-7(8H)-one or a pharmaceutically acceptable salt thereof.

12. The method of claim 11, wherein the cancer is selected from the group consisting of non-Hodgkin lymphoma (NHL), B-cell lymphomas, including Diffuse Large B-call Lymphoma (DLBCL), Mantle Cell Lymphoma (MCL), Burkitt's Lymphoma, Follicular Lymphoma (FL), Marginal Zone Lymphomas (MZL), Chronic Lymphocytic Leukemia/Small Lymphocytic Lymphoma, and Lymphoplasmacytic Lymphoma.

13. The method of claim 12, wherein the cancer is selected from the group consisting of relapsed or refractory indolent B-cell Non-Hodgkin Lymphoma, Mantle Cell Lymphoma and Chronic Lymphocytic Leukemia.

14. The method of claim 13, wherein the compound of Formula IA is administered in combination with bendamustine.

15. The method of claim 14, wherein the compound of Formula IA is administered in combination with rituximab.

16. The method of claim 15, wherein the compound of Formula IA is administered in combination with bendamustine and rituximab.

17. A method of treating a hematologic malignancy in a patient, comprising administering to the patient an effective amount of (a) 2-amino-8-ethyl-4-methyl-6-(1H-pyrazol-5-yl)pyrido[2,3-d]pyrimidin-7(8H)-one or a pharmaceutically acceptable salt thereof, and either (b) bendamustine or a pharmaceutically acceptable salt thereof or (c) rituximab or (d) a combination of bendamustine or a pharmaceutically acceptable salt thereof and rituximab, wherein the method comprises at least one cycle, wherein the cycle is a period of 28 days,
wherein 2-amino-8-ethyl-4-methyl-6-(1H-pyrazol-5-yl)pyrido[2,3-d]pyrimidin-7(8H)-one or the pharmaceutically acceptable salt thereof is administered at about 30 mg BID to about 50 mg BID, and wherein bendamustine or the pharmaceutically acceptable salt thereof is administered at about 70 mg/m² to about 90 mg/m², and rituximab is administered at about 375 mg/m² to about 500 mg/m²,
wherein the hematologic malignancy is relapsed or refractory indolent B-cell Non-Hodgkin Lymphoma, Mantle Cell Lymphoma or Chronic Lymphocytic Leukemia.

18. The method of claim 17, wherein rituximab is administered intravenously weekly for four to eight weeks.

19. The method of claim 18, wherein rituximab is administered on days 1, 8, 15 and 28 of the cycle.

20. The method of claim 17, wherein bendamustine or the pharmaceutically acceptable salt thereof is administered at about 90 mg/m² intravenously on days 1 and 2 of the cycle for up to eight cycles.

21. The method of claim 17, wherein bendamustine or the pharmaceutically acceptable salt thereof is administered at about 70 mg/m² intravenously on days 1 and 2 of the cycle for up to six cycles.

22. The method of claim 17, wherein rituximab is administered intravenously on day 1 and bendamustine or the pharmaceutically acceptable salt thereof is administered at about 90 mg/m² intravenously on days 1 and 2 of the cycle for up to eight cycles.

23. The method of claim 17, wherein rituximab is administered at about 375 mg/m² on day 1 of cycle 1 and at about 500 mg/m² on day 1 of cycles 2-6, and wherein bendamustine or the pharmaceutically acceptable salt thereof is administered at about 70 mg/m² on days 1 and 2 of the cycle for up to six cycles.

24. The method of claim 16 or 23, wherein the effective amount produces at least one therapeutic effect selected from the group consisting of reduction in size of a tumor, reduction in metastasis, complete remission, partial remission, stable disease, increase in overall response rate, or a pathologic complete response.

25. A combination for use in treating a hematologic malignancy, the combination comprising a therapeutically effective amount of (a) 2-amino-8-ethyl-4-methyl-6-(1H-pyrazol-5-yl)pyrido[2,3-d]pyrimidin-7(8H)-one or a pharmaceutically acceptable salt thereof, and either (b) bendamustine or a pharmaceutically acceptable salt thereof or (c) rituximab or (d) a combination of bendamustine or a pharmaceutically acceptable salt thereof and rituximab.

26. The combination of claim 25, wherein 2-amino-8-ethyl-4-methyl-6-(1H-pyrazol-5-yl)pyrido[2,3-d]pyrimidin-7(8H)-one or the pharmaceutically acceptable salt thereof is administered at about 30 mg BID to about 50 mg BID, and wherein bendamustine or the pharmaceutically acceptable salt thereof is administered at about 70 mg/m² to about 90 mg/m², and rituximab is administered at about 375 mg/m² to about 500 mg/m², and wherein the hematologic malignancy is relapsed or refractory indolent B-cell Non-Hodgkin Lymphoma, Mantle Cell Lymphoma or Chronic Lymphocytic Leukemia.
The method of claim 17, wherein rituximab is administered intravenously weekly for four to eight weeks.

27. The combination of claim 26, wherein rituximab is administered on days 1, 8, 15 and 28 of the cycle.

28. The combination of claim 26, wherein bendamustine or the pharmaceutically acceptable salt thereof is administered at about 90 mg/m² intravenously on days 1 and 2 of the cycle for up to eight cycles.

29. The combination of claim 25, wherein bendamustine or the pharmaceutically acceptable salt thereof is administered at about 70 mg/m² intravenously on days 1 and 2 of the cycle for up to six cycles.

30. The combination of claim 29, wherein rituximab is administered intravenously on day 1 and bendamustine or the pharmaceutically acceptable salt thereof is administered at about 90 mg/m² intravenously on days 1 and 2 of the cycle for up to eight cycles.

31. The combination of claim 30, wherein rituximab is administered at about 375 mg/m² on day 1 of cycle 1 and at about 500 mg/m² on day 1 of cycles 2-6, and wherein bendamustine or the pharmaceutically acceptable salt thereof is administered at about 70 mg/m² on days 1 and 2 of the cycle for up to six cycles.

32. The combination of claim 31, wherein the effective amount produces at least one therapeutic effect selected from the group consisting of reduction in size of a tumor, reduction in metastasis, complete remission, partial remission, stable disease, increase in overall response rate, or a pathologic complete response.
